(19) United States
(12) Patent Application Publication
(10) Pub. No.: US 2024/0167081 A1
(43) Pub. Date: May 23, 2024

(54) IMMOBILIZATION METHODS AND COMPOSITIONS FOR IN SITU DETECTION

(71) Applicant: 10x Genomics,Inc., Pleasanton, CA (US)

(72) Inventors: Felice Alessio BAVA, Rome (IT); Cheyenne CHRISTOPHERSON, San Ramon, CA (US); Justin COSTA, Union City, CA (US); Shalini GOHIL, Wellesley, MA (US); Christina GALONSKA, Christina (SE); Monica NAGENDRAN, Pleasanton, CA (US)

(21) Appl. No.: 18/503,949

(22) Filed: Nov. 7, 2023

Related U.S. Application Data
(60) Provisional application No. 63/423,750, filed on Nov. 8, 2022.

(51) Int. Cl. C12Q 1/682; C12Q 1/6841; C12Q 1/6844; C12Q 1/6876; G01N 1/30

(52) U.S. Cl. CPC C12Q 1/682; C12Q 1/6841; C12Q 1/6844; C12Q 1/6876; G01N 1/30

(57) ABSTRACT

The present disclosure relates in some aspects to methods and compositions for immobilization of nucleic acids for in situ detection in a biological sample or a matrix embedding the biological sample.

### CROSS-REFERENCE TO RELATED APPLICATIONS

This application claims priority to U.S. Provisional Patent Application No. 63/423,750, filed Nov. 8, 2022, entitled “IMMOBILIZATION METHODS AND COMPOSITIONS FOR IN SITU DETECTION” which is herein incorporated by reference in its entirety for all purposes.

### FIELD

The present disclosure relates in some aspects to methods and compositions for analysis of a target nucleic acid in a sample (e.g., in situ), such as analysis using oligonucleotides comprising crosslinkable nucleotides.

### BACKGROUND

Oligonucleotide probe-based assay methods for analysis of target nucleic acids depend on careful optimization related to the stability of the hybridization complex and/or the positional stability of the hybridization complex. For example, if the wash conditions are too stringent, then probe/target hybrids or amplification products thereof will be denatured, resulting in a decrease in the amount of signal in the assay. Furthermore, some methods such as isometric expansion of a sample require stabilization of target analytes to a matrix in order to preserve positional information of the target analytes in the sample (e.g., a cell or tissue sample). Thus, there is a need for increasing the spatial fidelity of target analytes (e.g., present in amplification products, such as rolling circle amplification products) during analysis of target nucleic acids in a sample (e.g., in situ analysis). Provided herein are methods and compositions that address such and other needs.

### SUMMARY

In some aspects, provided herein are immobilization oligonucleotides and probe designs that are useful for anchoring target analytes, probes, and/or labeling agents to preserve their spatial fidelity in a biological aspects. In some aspects, the compositions and methods provided herein include new and improved methods for anchoring target analytes such as RNA in a biological sample, particularly for biological samples containing fragmented RNA such as formalin-fixed, paraffin-embedded biological samples.

In some aspects, provided herein is a method, comprising: (a) contacting a biological sample with an immobilization oligonucleotide functionalized with a crosslinkable moiety and an attachment moiety, wherein the immobilization oligonucleotide comprises a hybridization region that hybridizes to a target nucleic acid in the biological sample; and (b) crosslinking the crosslinkable moiety of the immobilization oligonucleotide to the hybridized target nucleic acid and attaching the attachment moiety to the biological sample or a matrix embedding the biological sample, thereby immobilizing the target nucleic acid in the biological sample or the matrix.

In some embodiments, the method comprises (c) hybridizing a primary probe or probe set to a target sequence in the target nucleic acid. In some embodiments, the method comprises (d) detecting the primary probe or probe set or a product of the primary probe or probe set associated with the target nucleic acid.

In any of the preceding embodiments, the hybridization region can comprise a sequence of at least 5, 10, 15, or 20 thymines. In some embodiments, the hybridization region is an oligo deoxythymidine (oligo dT) sequence.

In any of the preceding embodiments, the target nucleic acid can be RNA. In any of the preceding embodiments, the target nucleic acid can be mRNA. In any of the preceding embodiments, the target nucleic acid can be an mRNA comprising a polyA tail. In any of the preceding embodiments, and the immobilization oligonucleotide may hybridize to the target nucleic acid at the polyA tail. In any of the preceding embodiments, the method can comprise hybridizing multiple copies of the immobilization oligonucleotide to the polyA tail.

In any of the preceding embodiments, wherein the target nucleic acid is an RNA fragment. In any of the preceding embodiments, the RNA fragment can be an mRNA fragment. In any of the preceding embodiments, the RNA fragment may be an RNA fragment that does not comprise a polyA tail.

In any of the preceding embodiments, the hybridization region can be a random sequence and/or comprises universal bases. In any of the preceding embodiments, the hybridization region can be a sequence of universal bases. In any of the preceding embodiments, the method may comprise hybridizing multiple immobilization oligonucleotides to the target nucleic acid.

In any of the preceding embodiments, the immobilization oligonucleotide may be configured to not be capable of being extended by a polymerase. In any of the preceding embodiments, the immobilization oligonucleotide may comprise a 3′ dideoxynucleotide.

In any of the preceding embodiments, the crosslinkable moiety can be a modified nucleoside in the immobilization oligonucleotide or can be connected to a nucleotide residue in the hybridization region of the immobilization oligonucleotide.

In any of the preceding embodiments, the crosslinking can occur between the hybridization region of the immobilization oligonucleotide and the hybridized target nucleic acid. In any of the preceding embodiments, the crosslinkable moiety can be configured to crosslink to a nucleobase of the hybridized target nucleic acid.

In any of the preceding embodiments, the method can comprises irradiating the biological sample or the matrix to photo-activate the crosslinkable moiety. In any of the preceding embodiments, the biological sample or the matrix can be irradiated using a 350-400 nm wavelength of light. In any of the preceding embodiments, the nucleobase can be a thymine, uridine, or cytosine. In any of the preceding embodiments, the nucleobase can an adenine.

In any of the preceding embodiments, the crosslinkable moiety can be connected to the nucleotide residue via a linker.

In any of the preceding embodiments, the crosslinkable moiety can be a vinylcarbazone-based moiety. In any of the preceding embodiments, the crosslinkable moiety can be a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D). In any of the preceding embodiments, the crosslinkable moiety can be a 3-cyanovinylcarbazole phosphoramidite or a pyranocarbazole phosphoramidite. In any of the preceding embodiments, the crosslinkable moiety can be a psoralen or a psoralen derivative. In some embodiments, the psoralen is a C2 psoralen. In some embodiments, the crosslinkable moiety is a psoralen C2 phosphoramidite. In any of the preceding embodiments, the crosslinkable moiety can be a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In any of the preceding embodiments, the crosslinkable moiety can be a 5′-Dimethoxytrityl-5-iodo-2′-deoxyUridine,3′-[(2-cyanoethyl)-(N,N-diisopropyl)]-phosphoramidite (5-I-dU-CE phosphoramidite).

In any of the preceding embodiments, the immobilization oligonucleotide can comprise two or more nucleotide residues functionalized with crosslinkable moieties in the hybridization region.

In any of the preceding embodiments, the immobilization oligonucleotide comprises three, four, five, or more nucleotide residues functionalized with crosslinkable moieties in the hybridization region. In any of the preceding embodiments, the hybridization region can comprise one or more universal bases. In any of the preceding embodiments, the nucleotide residue comprising or connected to the crosslinkable moiety can comprise a universal base. In any of the preceding embodiments, the nucleotide residues connected to the crosslinkable moieties can comprise universal bases.

In any of the preceding embodiments, the one or more universal bases can comprise a pseudouridine and/or an inosine. In some embodiments, the universal base is pseudouridine.

In any of the preceding embodiments, the attachment moiety can be an amine, a thiol, an azide, an alkyne, a nitrone, an alkene, a tetrazine, a tetrazole, an acrydite or other click reactive group.

In any of the preceding embodiments, the attachment moiety can be attached to an anchoring moiety in the biological sample or the matrix, wherein the attachment moiety and the anchoring moiety are a ligand-ligand binding pair, or functional moieties that can react with each other. In any of the preceding embodiments, the attachment moiety can be an acrydite moiety. In any of the preceding embodiments, the acrydite can be a C6 methacrylate. In any of the preceding embodiments, the attachment moiety can be a methacrylate C6 phosphoramidite.

In any of the preceding embodiments, the primary probe or probe set can be a circular probe or a circularizable probe or probe set. In any of the preceding embodiments, the method can comprise circularizing the circularizable probe or probe set to generate a circularized probe. In any of the preceding embodiments, the method can comprise performing rolling circle amplification of the circular or circularized probe to generate a rolling circle amplification product (RCP). In any of the preceding embodiments, the rolling circle amplification can be performed using a primer comprising a functional moiety for attachment to the biological sample or the matrix. In any of the preceding embodiments, the functional moiety of the primer can be orthogonal to the attachment moiety of the immobilization oligonucleotide. In any of the preceding embodiments, the method can comprise contacting the biological sample or the matrix with a nucleotide mixture comprising one or more modified crosslinkable nucleotides for incorporation into the RCP. In any of the preceding embodiments, the method can comprise crosslinking the functional moiety of the primer and/or the one or more modified crosslinkable nucleotide residues in the RCP to the biological sample or the matrix.

In any of the preceding embodiments, the detecting in (d) can comprise detecting the RCP. In any of the preceding embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In any of the preceding embodiments, the method can comprise performing one or more wash steps to remove unbound and/or nonspecifically bound intermediate probe molecules from the primary probes or the products of the primary probes.

In any of the preceding embodiments, the detecting in (d) can comprise: detecting signals associated with detectably labeled probes that are hybridized to barcode regions or complements thereof in the primary probe or probe set or a product thereof; and/or detecting signals associated with detectably labeled probes that are hybridized to intermediate probes which are in turn hybridized to the barcode regions or complements thereof. In any of the preceding embodiments, the detectably labeled probes can be fluorescently labeled.

In any of the preceding embodiments, the detecting in (d) can comprise binding an intermediate probe directly or indirectly to the primary probe or probe set, binding a detectably labeled probe directly or indirectly to a detection region of the intermediate probe, and detecting a signal associated with the detectably labeled probe.

In any of the preceding embodiments, detecting the primary probe or probe set can comprise amplifying a signal associated with the primary probe or probe set, wherein amplifying the signal comprises RCA of a probe that directly or indirectly binds to the primary probe or probe set and/or the amplification product thereof; hybridization chain reaction (HCR) directly or indirectly on the primary probe or probe set and/or the amplification product thereof; linear oligonucleotide hybridization chain reaction (LO-HCR) directly or indirectly on the primary probe or probe set and/or the amplification product thereof; primer exchange reaction (PER) directly or indirectly on the primary probe or probe set and/or the amplification product thereof; assembly of branched structures directly or indirectly on the primary probe or probe set and/or the amplification product thereof; hybridization of a plurality of detectable probes directly or indirectly on the primary probe or probe set and/or the amplification product thereof, or any combination thereof.

In any of the preceding embodiments, the method can comprise contacting the sample with a matrix-forming material and using the matrix-forming material to form the matrix. In any of the preceding embodiments, the matrix can be a hydrogel matrix. In any of the preceding embodiments, the matrix can be functionalized with the anchoring moiety to bind covalently or non-covalently to the attachment moiety. In any of the preceding embodiments, the anchoring moiety can be a reactive group selected from the group consisting of acrydite, NHS ester, azide, maleimide, amine, and carboxyl groups.

In any of the preceding embodiments, the attachment moiety can be a first attachment moiety, and the immobilization oligonucleotide can comprise a second attachment moiety. In some embodiments, the method comprises attaching the second attachment moiety to the biological sample or a matrix embedding the biological sample. In some embodiments, the second attachment moiety is different from the first attachment moiety. In any of the preceding embodiments, the second attachment moiety can be an amine, a thiol, an azide, an alkyne, a nitrone, an alkene, a tetrazine, a tetrazole, an acrydite or other click reactive group. In any of the preceding embodiments, the second attachment moiety can be a photo-crosslinkable nucleotide. In any of the preceding embodiments, the second attachment moiety is 5-bromo deoxyuridine (BrdU). In any of the preceding embodiments, the second attachment moiety can be a psoralen. In any of the preceding embodiments, the second attachment moiety can be attached to a second anchoring moiety in the biological sample or a matrix embedding the biological sample, wherein the second attachment moiety and the second anchoring moiety are a ligand-ligand binding pair, or functional moieties that can react with each other. In any of the preceding embodiments, the first attachment moiety can be attached to the matrix embedding the biological sample, and the second attachment moiety can be attached to the biological sample (e.g., to a protein in the biological sample). In any of the preceding embodiments, the first attachment moiety and the second attachment moiety can be attached to the same matrix embedding the biological sample using orthogonal reaction chemistries. In any of the preceding embodiments, the first attachment moiety can be attached to a first matrix embedding the biological sample and the second attachment moiety can be attached to a second matrix embedding the biological sample. In some embodiments, the first and second matrix are intertwined. In some embodiments, the first and second matrix are covalently or non-covalently attached to each other. In some embodiments, the first and second matrix are not attached to each other.

In some aspects, provided herein is a method of analyzing a biological sample, comprising: (a) performing an extension reaction of a primary probe hybridized to a target nucleic acid in the biological sample to incorporate one or more nucleotides functionalized with an attachment moiety using the target nucleic acid as a template, thereby forming a primary immobilizable probe; (b) attaching the attachment moiety to the biological sample or a matrix embedding the biological sample; and (c) detecting the attached primary immobilizable probe at a position in the biological sample or the matrix. In any of the preceding embodiments, the primary probe can comprise an overhang region at its 5′ end. In any of the preceding embodiments, the method can comprise performing extension reactions of a plurality of primary probes hybridized to the target nucleic acid. In any of the preceding embodiments, the extension reactions can be to incorporate one or more nucleotides functionalized with an attachment moiety using the target nucleic acid as a template into the plurality of primary probes. In some embodiments, the attachment moiety is a crosslinkable moiety. In any of the preceding embodiments, each primary probe of the plurality of primary probes can comprise an overhang region at its 5′ end.

In any of the preceding embodiments, the method can comprise hybridizing a detection probe to the overhang region and detecting the detection probe or a product thereof. In any of the preceding embodiments, the detection probe can comprise (i) a recognition sequence that hybridizes to a sequence of the overhang region and (ii) a reporter sequence for binding directly or indirectly to a detectably labeled probe. In any of the preceding embodiments, the detection probe can be a first detection probe, and the method comprises removing the first detection probe after detecting the first detection probe, hybridizing a second detection probe to the overhang region, and detecting the second detection probe. In any of the preceding embodiments, detection probe can be a circular or circularizable probe, and the method can comprise performing rolling circle amplification of the detection probe and detecting a product thereof. In any of the preceding embodiments, the detection probe can be a detectably labeled probe. In any of the preceding embodiments, the extension reactions can be performed simultaneously. In any of the preceding embodiments, the extension reaction or extension reactions can be performed using a polymerase lacking strand displacing activity. In some embodiments, the extension reaction of a primary probe of the plurality of primary probes does not displace other primary probes of the plurality of primary probes from the target nucleic acid. In any of the preceding embodiments, the extension reaction or extension reactions may be performed for less than 30 minutes, less than 10 minutes, or less than 5 minutes.

In some aspects, provided herein is a method of analyzing a biological sample, comprising: (a) contacting the biological sample with a primary probe; (b) hybridizing a secondary immobilizable probe to the primary probe to form an immobilizable probe complex, wherein the secondary immobilizable probe is functionalized with a crosslinkable moiety and an attachment moiety, (c) crosslinking the crosslinkable moiety of the immobilization oligonucleotide to the hybridized primary probe and attaching the attachment moiety to the biological sample or a matrix embedding the biological sample, thereby forming a crosslinked probe complex, and (d) hybridizing a detection probe to the primary probe and detecting the detection probe or a product thereof, thereby detecting the crosslinked probe complex at a position in the biological sample or the matrix. In some embodiments, (b) comprises hybridizing a plurality of secondary immobilizable probes to the primary probe to form the immobilizable probe complex, wherein each secondary immobilizable probe comprises an attachment moiety, and the method comprises, using the attachment moiety, attaching each secondary immobilizable probe to the biological sample or the matrix to form the crosslinked probe complex. In any of the preceding embodiments, the secondary immobilizable probe or plurality thereof can hybridize to an overhang region of the primary probe. In any of the preceding embodiments, the secondary immobilizable probe or plurality thereof can be a probe that does not comprise a detectable label. In any of the preceding embodiments, the detection probe can comprise (i) a recognition sequence that hybridizes to a sequence of the overhang region of the primary probe, and (ii) a reporter sequence for binding directly or indirectly to a detectably labeled probe. In any of the preceding embodiments, the detection probe can be a first detection probe, and the method can comprise removing the first detection probe after detecting the first detection probe, hybridizing a second detection probe to the overhang region, and detecting the second detection probe. In any of the preceding embodiments, the detection probe can be a circular or circularizable probe, and the method can comprise performing rolling circle amplification of the detection probe (e.g., a circularized probe formed from the circularizable probe) and detecting a product thereof (e.g., a rolling circle amplification product of the detection probe). In any of the preceding embodiments, the detection probe can be a detectably labeled probe.

In any of the preceding embodiments, the method can further comprise hybridizing a tertiary immobilizable probe comprising an attachment moiety to the secondary probe or plurality thereof. In any of the preceding embodiments, the method can further comprise crosslinking the tertiary immobilizable probe to the biological sample or the matrix to form the crosslinked probe complex. In any of the preceding embodiments, the crosslinkable moiety can be a modified nucleoside in the immobilization oligonucleotide or is connected to a nucleotide residue in the hybridization region of the immobilization oligonucleotide. In any of the preceding embodiments, the crosslinking can occur between the hybridization region of the immobilization oligonucleotide and the hybridized target nucleic acid. In any of the preceding embodiments, the crosslinkable moiety can be configured to crosslink to a nucleobase of the hybridized target nucleic acid.

In any of the preceding embodiments, the method can comprise irradiating the biological sample or the matrix to photo-activate the crosslinkable moiety. In any of the preceding embodiments, the biological sample or the matrix can be irradiated using a 350-400 nm wavelength of light. In any of the preceding embodiments, the nucleobase can be a thymine, uridine, or cytosine. In any of the preceding embodiments, the nucleobase can be an adenine.

In any of the preceding embodiments, the crosslinkable moiety can be connected to the nucleotide residue via a linker. In any of the preceding embodiments, the crosslinkable moiety can be a vinylcarbazone-based moiety. In any of the preceding embodiments, the crosslinkable moiety can be a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D). In any of the preceding embodiments, the crosslinkable moiety can be a 3-cyanovinylcarbazole phosphoramidite or a pyranocarbazole phosphoramidite. In any of the preceding embodiments, the crosslinkable moiety can be a psoralen or a psoralen derivative, optionally wherein the psoralen is a C2 psoralen. In any of the preceding embodiments, the crosslinkable moiety can be a psoralen C2 phosphoramidite. In any of the preceding embodiments, the crosslinkable moiety can be a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In any of the preceding embodiments, the crosslinkable moiety can be a 5′-Dimethoxytrityl-5-iodo-2′-deoxyUridine,3′-[(2-cyanoethyl)-(N,N-diisopropyl)]-phosphoramidite (5-I-dU-CE phosphoramidite). In any of the preceding embodiments, the immobilization oligonucleotide can comprise two or more nucleotide residues functionalized with crosslinkable moieties in the hybridization region. In any of the preceding embodiments, the immobilization oligonucleotide can comprise three, four, five, or more nucleotide residues functionalized with crosslinkable moieties in the hybridization region.

In any of the preceding embodiments, the hybridization region can comprise one or more universal bases. In any of the preceding embodiments, the nucleotide residue comprising or connected to the crosslinkable moiety can comprise a universal base. In any of the preceding embodiments, the nucleotide residues connected to the crosslinkable moieties can comprise universal bases.

In any of the preceding embodiments, the secondary immobilizable probe or plurality thereof can each comprise a universal hybridization region comprising (i) one or more universal or random bases and (ii) the crosslinkable moiety. In any of the preceding embodiments, the universal hybridization region of the secondary immobilizable probe or plurality thereof can hybridize non-specifically to the primary probe. In any of the preceding embodiments, the primary probe can be hybridized to a target nucleic acid in the sample. In any of the preceding embodiments, the primary probe can be crosslinked to the target nucleic acid, to the biological sample, and/or to the matrix. In some embodiments, the primary probe is crosslinked to the target nucleic acid, to the biological sample (e.g., to a protein in the biological sample), and to the matrix.

In some aspects, provided herein is a method of analyzing a tissue sample, comprising: (a) contacting the tissue sample with a primary immobilizable probe, wherein the primary immobilizable probe comprises a hybridization region capable of hybridizing to a region of interest in a target nucleic acid and an attachment moiety; (b) using the attachment moiety, crosslinking the primary immobilizable probe a matrix embedding the biological sample; (c) clearing the tissue sample; (d) hybridizing a detection probe to a barcode sequence in the primary immobilizable probe; and (e) detecting the detection probe or a product thereof at a position in the biological sample or the matrix. the detection probe comprises (i) a recognition sequence that hybridizes to a sequence of the overhang region of the primary probe, and (ii) a reporter sequence for binding directly or indirectly to a detectably labeled probe. In any of the preceding embodiments, the detection probe can be a first detection probe, and the method can comprise removing the first detection probe after detecting the first detection probe, hybridizing a second detection probe to the overhang region, and detecting the second detection probe. In any of the preceding embodiments, the detection probe can be a circular or circularizable probe, and the method can comprise performing rolling circle amplification of the detection probe and detecting a product thereof (e.g., the rolling circle amplification product of the detection probe). In any of the preceding embodiments, the detection probe can be a detectably labeled probe.

In some aspects, provide herein is a method of analyzing a biological sample, comprising: (a) contacting the biological sample with an immobilization oligonucleotide and a primary probe, wherein the primary probe hybridizes to a target nucleic acid in the biological sample, and wherein the immobilization oligonucleotide comprises an attachment moiety; (b) ligating the primary probe to the immobilization oligonucleotide to form a ligated immobilizable probe comprising the primary probe and the immobilization oligonucleotide; (c) crosslinking the attachment moiety of the ligated immobilizable probe to a matrix embedding the biological sample, thereby crosslinking the immobilizable probe to the matrix; (d) contacting the biological sample with a detection probe that hybridizes to the ligated immobilizable probe or a product thereof; and (e) detecting the detection probe or a product of the detection probe at a position in the biological sample. In any of the preceding embodiments, the detection probe can hybridize to a detection probe hybridization sequence in a first overhang region of the primary probe in the immobilizable probe.

In any of the preceding embodiments, the method can comprise contacting the immobilization oligonucleotide with a splint that hybridizes to at least a portion of the primary probe and at least a portion of the immobilization oligonucleotide. In any of the preceding embodiments, the splint can serve as a template for ligating the primary probe to the immobilization oligonucleotide. In any of the preceding embodiments, the splint can hybridize to a splint hybridization sequence in a second overhang region of the primary probe. In any of the preceding embodiments, the immobilization oligonucleotide and the primary probe can hybridize to adjacent sequences of the target nucleic acid. In any of the preceding embodiments, the target nucleic acid can serve as a template for ligating the primary probe to the immobilization oligonucleotide.

In some aspects, provided herein is a method of analyzing a biological sample, comprising: (a) contacting the biological sample with a probe or probe set comprising a first hybridization region and a second hybridization region, wherein the first hybridization region and the second hybridization region hybridize to a first and second target sequence, respectively, in a target nucleic acid, wherein the first and second target sequences are 3′ and 5′, respectively, to a first sequence of a region of interest in the target nucleic acid, wherein the region of interest comprises a first nucleobase; (b) contacting the biological sample with a crosslinkable nucleotide complementary to the first nucleobase; (c) extending the 3′ end of the first hybridization region with a polymerase using the first sequence of the region of interest as a template, thereby incorporating the crosslinkable nucleotide into the first hybridization region; (d) ligating the extended 3′ end of the first hybridization region and the 5′ end of the second hybridization region to form a ligated probe; (e) crosslinking the incorporated crosslinkable nucleotide to the biological sample or a matrix embedding the biological sample; and (f) detecting the crosslinked ligated probe or a product thereof at a location in the biological sample or the matrix. In any of the preceding embodiments, the ligatable probe or probe set can be a ligatable probe set comprising a first part and a second part, wherein the first part comprises the first hybridization region and the second part comprises the second hybridization region. In any of the preceding embodiments, detecting the crosslinked ligated probe can comprise detecting a sequence in an overhang region of the first part and/or second part. In any of the preceding embodiments, the ligatable probe or probe set is a circularizable probe or probe set.

In some aspects, provided herein is a method of analyzing a biological sample, comprising: (a) contacting the biological sample with a circularizable probe comprising (i) a 3′ arm that hybridizes to a first target sequence in a target nucleic acid in the biological sample, and (ii) a 5′ arm that hybridizes to a second target sequence in the target nucleic acid, wherein the first and second target sequence are 3′ and 5′, respectively, to a first sequence of a region of interest comprising a first nucleobase; (b) contacting the biological sample with a crosslinkable nucleotide complementary to the first nucleobase; (c) extending the 3′ arm of the circularizable probe with a polymerase using the first sequence of the region of interest as a template, thereby incorporating the crosslinkable nucleotide into the circularizable probe; (d) ligating the extended 3′ arm and the 5′ arm of the circularizable probe to form a circularized probe; (e) crosslinking the incorporated crosslinkable nucleotide to the biological sample or to a matrix embedding the biological sample; and (f) detecting the crosslinked circularized probe or a product thereof at a location in the biological sample or the matrix. In any of the preceding embodiments, the biological sample can comprise an alternative sequence of the region of interest that does not comprise the first nucleobase, such that the crosslinkable nucleotide is not incorporated into the circularizable probe when using the alternative sequence of the region of interest as a template. In any of the preceding embodiments, detecting the crosslinked circularized probe or a product thereof can comprise performing rolling circle amplification (RCA) using the circularized probe as a template to form a rolling circle amplification product (RCP) and detecting the RCP in the sample. In any of the preceding embodiments, the method can comprise decrosslinking the circularized probe prior to performing RCA.

In any of the preceding embodiments, the method can comprise (a) hybridizing a secondary circular probe to the crosslinked circularized probe, or hybridizing a secondary circularizable probe to the crosslinked circularized probe and circularizing the hybridized secondary circularizable probe to generate a secondary circularized probe, and (b) performing RCA using the secondary circular probe or secondary circularized probe as a template to form a rolling circle amplification product (RCP) and detecting the RCP in the sample. In any of the preceding embodiments, the target nucleic acid can be a cDNA.

In any of the preceding embodiments, the matrix-forming material can be a first species of matrix-forming material, and the method can comprise polymerizing the first species of matrix-forming material to form a first matrix, contacting the first matrix with a second species of matrix-forming material, and polymerizing the second species of matrix-forming material to form a second matrix. In any of the preceding embodiments, the method can comprise polymerizing the second species of matrix-forming material to form the second matrix after hybridizing a primary probe or probe set to a target sequence in the target nucleic acid. In any of the preceding embodiments, the method can comprise contacting the first matrix with the second species of matrix-forming material after hybridizing a primary probe or probe set to a target sequence in the target nucleic acid.

In any of the preceding embodiments, the matrix can be a hydrogel matrix. In any of the preceding embodiments, the first matrix and/or the second matrix can be a hydrogel matrix.

In any of the preceding embodiments, the biological sample can be non-homogenized. In any of the preceding embodiments, the biological sample can be selected from the group consisting of a formalin-fixed, paraffin-embedded (FFPE) sample, a frozen tissue sample, and a fresh tissue sample. In any of the preceding embodiments, the biological sample can be permeabilized. In any of the preceding embodiments, the biological sample can be embedded in a matrix. In any of the preceding embodiments, the matrix can comprise a hydrogel. In any of the preceding embodiments, the biological sample can be cleared. In any of the preceding embodiments, the biological sample can be cleared by a clearing step comprising contacting the biological sample with a proteinase. In any of the preceding embodiments, the method comprises clearing the biological sample after crosslinking the crosslinkable moiety to the target nucleic acid and attaching the attachment moiety to the matrix. In any of the preceding embodiments, the clearing can comprise contacting the biological sample with a proteinase. In any of the preceding embodiments, the biological sample can be a tissue slice between about 1 μm and about 50 μm in thickness. In some embodiments, the tissue slice is between about 5 μm and about 35 μm in thickness.

### BRIEF DESCRIPTION OF THE DRAWINGS

The drawings illustrate certain features and advantages of this disclosure. These embodiments are not intended to limit the scope of the appended claims in any manner.

FIG. 1A schematically depicts an example immobilization oligonucleotide functionalized with a crosslinkable moiety and an attachment moiety hybridized to a target nucleic acid.

FIG. 1B schematically depicts a plurality of target nucleic acids immobilized in a matrix by interstrand crosslinking between the target nucleic acids and the hybridized immobilization oligonucleotides and attachment of the attachment moiety of the immobilization moieties to the matrix.

FIG. 1C schematically depicts an example immobilization oligonucleotide functionalized with a psoralen crosslinkable moiety and a methacrylate attachment moiety.

FIGS. 2A-2C depict various example primary probe or probe sets and detection schemes.

FIG. 3A shows an example of a tri-functional immobilization oligonucleotide comprising a psoralen, a methacrylate, and a 5-bromo deoxyuridine.

FIG. 3B shows an example of a tri-functional immobilization oligonucleotide comprising a psoralen, a methacrylate, and a 5-octadiynyl moiety.

FIG. 4 illustrates an example of a workflow comprising contacting a biological sample with a first species of matrix forming material, forming a first matrix from the first species of matrix-forming material, contacting the biological sample with a second species of matrix-forming material, and forming a second matrix from the second-species of matrix forming material. Advantageously, different species of matrix-forming material can be used to anchor different attachment moieties to the matrices.

FIG. 5A depicts an embodiment of a method comprising immobilization of a primary probe by incorporation of one or more nucleotides functionalized with an attachment moiety, which can be crosslinked to a target nucleic acid hybridized by the primary probe, or to another molecule in the biological sample or a matrix embedding the biological sample. 3A: workflow for generation and detection of primary immobilizable probe

FIGS. 5B-5D depict various examples of detection schemes for an immobilized primary probe in a biological sample.

FIG. 6A depicts an embodiment of an immobilizable probe complex comprising a secondary immobilizable probe hybridized to a primary probe, wherein the primary probe is hybridized to a target nucleic acid.

FIG. 6B depicts an embodiment of an immobilizable probe complex comprising a secondary immobilizable probe hybridized to a primary probe, and a tertiary immobilizable probe bound to the secondary immobilizable probe.

FIGS. 6C-6E depict various examples of detection schemes for an immobilizable probe complex.

FIG. 7A-7B depict an example of a workflow comprising gap-fill incorporation of a crosslinkable nucleotide (depicted as Z′) complementary to a SNP in a region of interest to detect the SNP in a target nucleic acid. FIG. 7A illustrates an example where the SNP is present and the crosslinkable nucleotide is incorporated, and FIG. 7B illustrates an example where the SNP is absent and no crosslinkable nucleotide is incorporated.

FIG. 7C illustrates a method comprising hybridizing a secondary circularizable probe to a crosslinked circularized probe, ligating the secondary circularizable probe to form a secondary circularized probe, and performing RCA of the secondary circularized probe. In some embodiments, the RCA product of the secondary circularized probe is detected in order to detect the crosslinked circularized probe such as that illustrated in FIG. 7A.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods involving the use of immobilization oligonucleotides functionalized with an attachment moiety for attachment to a biological sample or a matrix (e.g., a 3D matrix such as a hydrogel), for immobilization and analysis of one or more target nucleic acid(s) (for example, a messenger RNA or analyte comprising a nucleic acid) present in a sample (e.g., a cell or a biological sample, such as a tissue sample). The methods relate, at least in some aspects, to covalently or non-covalently anchoring a target nucleic acid to the biological sample or a matrix using any one of the immobilization oligonucleotide designs disclosed herein, wherein the immobilization oligonucleotide hybridizes to the target nucleic acid. In some aspects, the immobilization oligonucleotides comprise a crosslinkable moiety for interstrand crosslinking to the target nucleic acid. Also provided are polynucleotides, sets of polynucleotides, compositions, kits, systems, and devices for use in accordance with the provided methods. In some aspects, the provided methods and compositions can be applied to maintain the spatial fidelity of the target nucleic acid during downstream analyses (e.g., in situ analysis).

### II. Polynucleotides and Hybridization Complexes

Disclosed herein in some aspects are nucleic acid probes and/or probe sets and immobilization oligonucleotides that are introduced into a cell or used to otherwise contact a biological sample such as a tissue sample. The probes may comprise any of a variety of entities that can hybridize to a nucleic acid, typically by Watson-Crick base pairing, such as DNA, RNA, LNA, PNA, etc., depending on the application. The nucleic acid probe(s) and immobilization oligonucleotides typically contains a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes may be detected using a detectable label, and/or by using detectably labeled nucleic acid probes able to bind to the nucleic acid probes or amplification products thereof, directly or via an intermediate probe. In some embodiments, the nucleic acid probes are compatible with one or more biological and/or chemical reactions. For instance, a primary nucleic acid probe disclosed herein can serve as a template or primer for a polymerase (e.g., for rolling circle amplification), a template or substrate for a ligase, a substrate for a click chemistry reaction, and/or a substrate for a nuclease (e.g., endonuclease for cleavage).

### A. Immobilization Oligonucleotides

In some aspects, provided herein are immobilization oligonucleotides for immobilizing target nucleic acids in a biological sample or matrix.

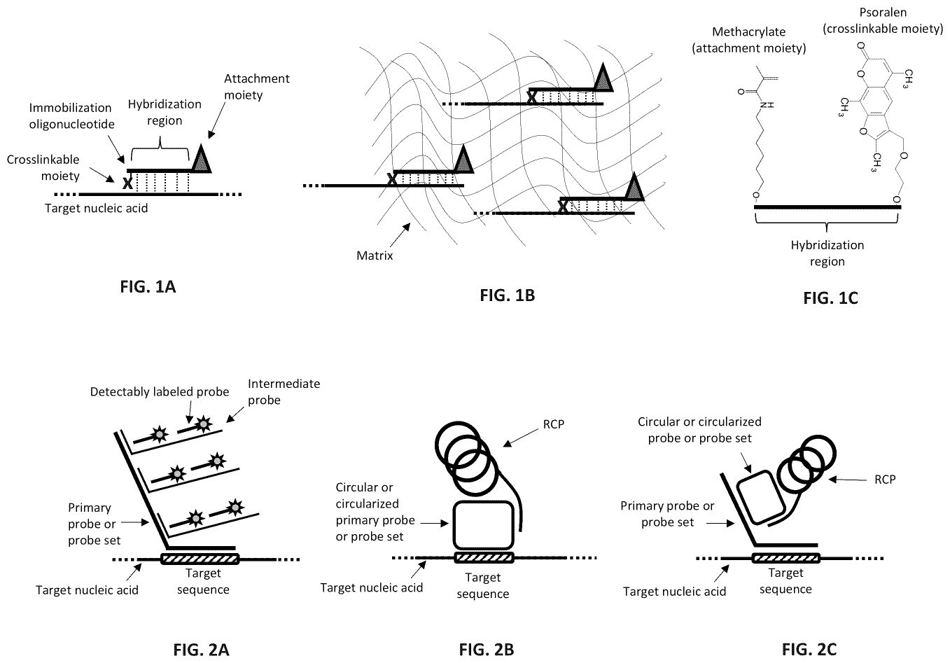

In some embodiments, an immobilization oligonucleotide provided herein is functionalized with a crosslinkable moiety and an attachment moiety. In some embodiments, the immobilization oligonucleotide comprises a hybridization region that hybridizes to a target nucleic acid in the biological sample. An example immobilization oligonucleotide is depicted schematically in FIG. 1B. The dashed lines between the hybridization region and the target nucleic acid depict hybridization to the target nucleic acid. In some cases, the hybridization region comprises a sequence complementary to a sequence in the target nucleic acid.

In some embodiments, the hybridization region is a sequence of thymines and/or uridines, which hybridizes to a complementary sequence of adenines. In some cases, the target nucleic acid is mRNA comprising a polyA tail, and the hybridization region hybridizes to the polyA tail. In some embodiments, the hybridization region is a sequence of between about 5 and about 60 thymines. In some embodiments, the hybridization region comprises a sequence of at least 5, 10, 15, or 20 thymines. In some embodiments, the hybridization region is an oligo deoxythymidine (oligo dT) sequence. In some instances, a method provided herein comprises hybridizing multiple copies of the immobilization oligonucleotide to the polyA tail of the mRNA. In some aspects, hybridization of the immobilization oligonucleotide to the polyA tail allows immobilization of a plurality of different target nucleic acids comprising polyA tails using the same species of immobilization oligonucleotide. In some embodiments, at least 2, 3, 4, 5, 10, or more immobilization oligonucleotides hybridize to the polyA tail. In some cases, the at least 2, 3, 4, 5, 10, or more immobilization oligonucleotides are crosslinked to the mRNA at multiple sites by forming an interstrand crosslink using the crosslinkable moiety. The multiple immobilization oligonucleotides can be attached to the biological sample or matrix via the attachment moieties of multiple immobilization oligonucleotides, thereby forming multiple attachments between the RNA and the biological sample or matrix.

In some embodiments, the hybridization region is a sequence of universal or random bases. In some aspects, the hybridization region comprising universal and/or random bases hybridizes non-specifically to the target nucleic acid (e.g., without sequence specificity). In some embodiments, the target nucleic acid in the biological sample is an RNA fragment, such as an mRNA fragment. In some cases, the immobilization oligonucleotide hybridizes non-specifically to the RNA fragment. In some instances, the RNA fragment does not comprise a polyA tail. Advantageously, in some aspects the hybridization region comprising universal and/or random bases allows immobilization of a plurality of RNA fragments in the biological sample or matrix, wherein the RNA fragments may not comprise a common sequence such as a polyA tail. In some embodiments, the multiple immobilization oligonucleotides hybridize to the target nucleic acid (e.g., via non-specific hybridization of a hybridization region comprising universal and/or random bases).

Example universal bases have been described, such as deoxyinosine (Ohtsuka, E. et al., (1985) J. Biol. Chem. 260, 2605-2608; and Sakanari, S. A. et al., (1989) Proc. Natl. Acad. Sci. 86, 4863-4867), 1-(2′-deoxy-beta-D-ribofuranosyl)-3-nitropyrrole (Nichols, R. et al., (1994) Nature 369, 492-493) and 5-nitroindole (Loakes, D. et al., (1994) Nucleic Acids Res. 22, 4039-4043). In some embodiments, the one or more universal bases of the hybridization region in the immobilization oligonucleotide comprise deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′O-methoxyethyl nebularine, 2′-O-methoxyethyl 5-nitroindole, 2′-O-methoxyethyl 4-nitrobenzimidazole, 2′-O-methoxyethyl 3-nitropyrrole, and combinations thereof. In some embodiments, the one or more universal bases of the hybridization region in the immobilization oligonucleotide comprise deoxyinosine, 1-(2′-deoxy-beta-D-ribofuranosyl)-3-nitropyrrole or 5-nitroindole. In some embodiments, the one or more universal bases of the hybridization region in the immobilization oligonucleotide can comprise deoxyinosine. In some cases, the universal bases can be pseudouridines or inosines. In some cases, the universal bases are pseudouridines. The universal bases can be connected to psoralens or other sequence-independent crosslinkable moieties via a linker. Other sequence independent crosslinkable moieties can include vinylcarbazone-based moieties. Vinylcarbazone-based moieties can include a cyanovinylcarbazole (^{CNV}K) nucleoside, a cyanovinylcarbazole modified D-threoninol (^{CNV}D), or a pyranocarbazole (^{PC}X) nucleoside, or a pyranocarbazole modified D-threoninol (^{PCX}D).

In some embodiments, the hybridization region is complementary to a sequence in the target nucleic acid. In some embodiments, the hybridization region is complementary to a sequence of a target nucleic acid of interest or a subset of target nucleic acids in the biological sample, whereby the target nucleic acid of interest or subset of target nucleic acids can be immobilized in the biological sample or matrix using the immobilization oligonucleotide.

In some embodiments, the hybridization region is between about 5 and about 60 nucleotides in length, e.g., between about 5 and about 50, between about 5 and about 40, between about 5 and about 30, between about 5 and about 25, between about 5 and about 15, between about 10 and about 50, between about 10 and about 40, between about 10 and about 30, or between about 20 and about 50 nucleotides in length.

As illustrated in FIG. 1B, in some embodiments, a plurality of target nucleic acids are immobilized in a matrix by interstrand crosslinking between the target nucleic acids and the hybridized immobilization oligonucleotides via the crosslinkable moiety, and attachment of the attachment moiety of the immobilization moieties to the matrix. In some embodiments, the crosslinking occurs between the hybridization region of the immobilization oligonucleotide and the hybridized target nucleic acid. In some embodiments, the crosslinkable moiety is configured to crosslink to a nucleobase of the hybridized target nucleic acid. In some embodiments, the crosslinkable moiety is a modified nucleoside in the immobilization oligonucleotide, or is connected to a nucleotide residue in the hybridization region of the immobilization oligonucleotide. Example crosslinkable moieties and attachment moieties are described in more detail in Sections III and IV below. In some embodiments, the crosslinkable moiety does not react with guanines of the target nucleic acid (e.g., the N7 position of guanines).

In some cases, the immobilization oligonucleotide comprises multiple (e.g., two, three, four, five, or more) nucleotide residues functionalized with crosslinkable moieties in the hybridization region. In some embodiments, the nucleotide residue or residues functionalized with one or more crosslinkable moieties comprise thymine or uridine bases (e.g., for hybridizing to a polyA sequence such as a polyA tail). In some embodiments, the nucleotide residue or residues functionalized with one or more crosslinkable moieties comprise universal and/or random bases (e.g., for hybridizing non-specifically to the target nucleic acid).

In some embodiments, the method does not comprise extending the immobilization oligonucleotide using a polymerase. In some embodiments, the immobilization oligonucleotide is configured to not be capable of being extended by a polymerase. In some instances, the immobilization oligonucleotide comprises a 3′ chain terminating group. In some instances, the immobilization oligonucleotide comprises a 3′ dideoxy group (e.g., a 3′ dideoxynucleotide).

In some embodiments, the immobilization oligonucleotide is bifunctional. In certain embodiments, a bifunctional immobilization oligonucleotide comprises (1) a crosslinkable moiety for interstrand crosslinking with a hybridized nucleic acid, and (2) an attachment moiety for attachment to a biological sample or a matrix. As illustrated in FIG. 1C, an example immobilization oligonucleotide can comprise a psoralen crosslinkable moiety and a C6 methacrylate attachment moiety.

In some embodiments, the immobilization oligonucleotide is trifunctional. In certain embodiments, the immobilization oligonucleotide comprises three different functional groups for interstrand crosslinking with a hybridized nucleic acid and/or for attachment to a biological sample or a matrix. In some embodiments, the immobilization oligonucleotide comprises (1) a crosslinkable moiety for interstrand crosslinking with a hybridized nucleic acid, (2) a first attachment moiety for attachment to a biological sample or a matrix, and (3) a second attachment moiety for attachment to a biological sample or a matrix. In some embodiments, the crosslinkable moiety, the first attachment moiety, and the second attachment moieties are all different. In some cases, the crosslinkable moiety is a modified nucleoside in the immobilization oligonucleotide or is connected to a nucleotide residue in the hybridization region of the immobilization oligonucleotide. In some embodiments, the first attachment moiety and the second attachment moiety are each selected from the group consisting of an amine, a thiol, an azide, an alkyne, a nitrone, an alkene, a tetrazine, a tetrazole, an acrydite, and any other click reactive groups. In some embodiments, the first attachment moiety and the second attachment moiety react with different anchoring moieties in one or more matrices embedding the biological sample via orthogonal reaction chemistries. In some embodiments, modified nucleotide residues at the 5′ end of the immobilization oligonucleotide, an internal nucleotide residue, and at the 3′ end of the immobilization oligonucleotide comprise or are the crosslinkable moiety, the first attachment moiety, and the second attachment moiety. In some embodiments, the crosslinkable moiety is at the 5′ end or 3′ end of the immobilization oligonucleotide, the first attachment moiety is at the 5′ end or 3′ end of the immobilization oligonucleotide, and the second attachment moiety is or is attached to a modified nucleotide residue at an internal position in the immobilization oligonucleotide.

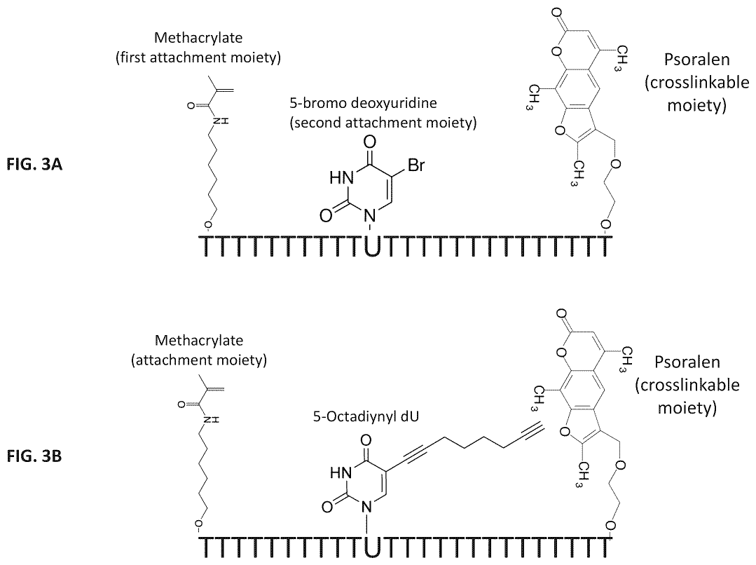

In some embodiments, the second attachment moiety is a photo-crosslinkable nucleoside such as a 5-bromo deoxyuridine (BrdU), or any other crosslinkable nucleoside described herein. 5-bromo-deoxyuridine is a photoreactive halogenated base that can be incorporated into oligonucleotides to crosslink them to DNA, RNA or proteins with exposure to UV light. Crosslinking is maximally efficient with light at 308 nm. 5-Bromo-deoxyuridine can readily be incorporated at internal positions of immobilization oligonucleotides. In some embodiments, an internal 5-Bromo-deoxyuridine allows cross reactivity with a hybridized nucleic acid, but also allows more options for crosslinking to the hydrogel. In some embodiments, the second attachment moiety is an internal nucleotide residue or is attached to an internal nucleotide residue in the immobilization oligonucleotide. A particular example of an immobilization oligonucleotide comprising a 3′ first attachment moiety (methacrylate), a 5′ crosslinkable moiety (psoralen), and an internal 5-bromo deoxyuridine (second attachment moiety) is provided in FIG. 3A). It will be understood that the first attachment moiety could be at the 3′ end or at an internal position of the immobilization oligonucleotide, and the crosslinkable moiety could be at the 5′ end or at an internal position of the immobilization oligonucleotide.

In some embodiments, the second attachment moiety is an amine, a thiol, an azide, an alkyne, a nitrone, an alkene, a tetrazine, a tetrazole, an acrydite or other click reactive group. In some cases, the second attachment moiety is an internal nucleotide residue or is attached to an internal nucleotide residue in the immobilization oligonucleotide. In some cases, the second attachment moiety is attached to an internal modified nucleotide residue using click chemistry. For example, in some cases, an internal nucleotide residue comprising a click chemistry group (e.g., 5-Octadiynyl dU) is used to click on any desired orthogonal chemistry moiety. In some cases, the immobilization oligonucleotide comprises a sequence of Ts and/or Us, wherein a 5′ end of the immobilization oligonucleotide comprises a first attachment moiety (methacrylate), an internal nucleobase in the immobilization oligonucleotide is 5-octadiynyl dU, and the 3′ end of the immobilization oligonucleotide comprises a psoralen crosslinkable moiety, such as in the particular embodiment shown in FIG. 3B. Click chemistry can also be used to click on any desired orthogonal chemistry moiety in the example immobilization oligonucleotide depicted in FIG. 3B. Additionally, it will be understood that the first attachment moiety could be at the 3′ end or at an internal position of the immobilization oligonucleotide, and the crosslinkable moiety could be at the 5′ end or at an internal position of the immobilization oligonucleotide.

In some embodiments, the trifunctional immobilization oligonucleotide comprises a crosslinkable moiety for interstrand crosslinking, a first attachment moiety, and a second attachment moiety. In some embodiments, the first attachment moiety is attached to a matrix embedding the biological sample, and the second attachment moiety is attached to the biological sample (e.g., by crosslinking the attachment moiety to a protein or nucleic acid in the biological sample). In some embodiments, the crosslinkable moiety is a 3-cyanovinylcarbazole (^{CNV}K) nucleoside crosslinkable moiety, the first attachment moiety is an acrydite moiety, and the second attachment moiety comprises a psoralen. In some embodiments, the acrydite moiety can be a C6 methacrylate. In some embodiments, the ^{CNV}K component of the trifunctional immobilization oligonucleotide is crosslinked to the target nucleic acid and the psoralen component of the trifunctional immobilization oligonucleotide is cross-linked to a protein in the biological sample. In some embodiments, the protein that is crosslinked to the psoralen component of the trifunctional immobilization oligonucleotide is in close proximity to the target nucleic acid. In some embodiments, the acrydite attachment moiety (e.g., C6 methacrylate) is reacted with acrylamide monomers in the matrix-forming material, thereby covalently attaching the matrix to the immobilization oligonucleotide. In some embodiments, the matrix forming material is a cleavable acrylamide type hydrogel. For example, the cleavable matrix forming material can be polyacrylamide gel cross-linker such as N,N′-(1,2-Dihydroxyethylene)bis-acrylamide. In some embodiments, following crosslinking of the two crosslinkable moieties (^{CNV}K and psoralen) to the target nucleic acid and a nearby protein in the biological sample, the of N′-(1,2-Dihydroxyethylene)bis-acrylamide can be cleaved with periodate, hence dissolving the acrylamide type hydrogel. Following dissolution of the hydrogel, the protein in the biological sample linked to psoralen can be analyzed.

In some embodiments, the trifunctional immobilization oligonucleotide comprises a psoralen crosslinkable moiety, a 3-cyanovinylcarbazole (^{CNV}K) nucleoside crosslinkable moiety, and an acrydite attachment moiety. In some embodiments, the acrydite attachment moiety can be a C6 methacrylate. In some embodiments, the ^{CNV}K component of the trifunctional immobilization oligonucleotide is crosslinked to the target nucleic acid and the psoralen component of the trifunctional immobilization oligonucleotide is cross-linked to a protein in the biological sample. In some embodiments, the protein that is crosslinked to the psoralen component of the trifunctional immobilization oligonucleotide is in close proximity to the target nucleic acid. In some embodiments, the acrydite attachment moiety (e.g., C6 methacrylate) is reacted with acrylamide monomers in the matrix-forming material, thereby covalently attaching the matrix to the immobilization oligonucleotide. In some embodiments, the matrix forming material is a cleavable acrylamide type hydrogel. For example, the cleavable matrix forming material can be polyacrylamide gel cross-linker such as N,N′-(1,2-Dihydroxyethylene)bis-acrylamide. In some embodiments, following crosslinking of the two crosslinkable moieties (^{CNV}K and psoralen) to the target nucleic acid and a nearby protein in the biological sample, the of N′-(1,2-Dihydroxyethylene)bis-acrylamide can be cleaved with periodate, hence dissolving the acrylamide type hydrogel. Following dissolution of the hydrogel, the protein in the biological sample linked to psoralen can be analyzed.

In some aspects, provided herein is a method of analyzing a biological sample, comprising contacting the biological sample with a primary probe, and hybridizing an immobilization oligonucleotide to the primary probe to form an immobilizable probe complex. In some embodiments, the immobilization oligonucleotide comprises an attachment moiety and a crosslinkable moiety. In some embodiments, the method further comprises crosslinking the crosslinkable moiety of the immobilization oligonucleotide to the hybridized primary probe and attaching the attachment moiety to the biological sample or a matrix embedding the biological sample, thereby forming a crosslinked probe complex. In some embodiments, the method further comprises hybridizing a detection probe to the primary probe and detecting the detection probe or a product thereof, thereby detecting the crosslinked probe complex at a position in the biological sample or matrix. In some embodiments, the primary probe is hybridized to a target nucleic acid in the sample. In some embodiments, the primary probe is crosslinked to the target nucleic acid, the biological sample, and/or the matrix.

In some aspects, the immobilizable probe complex is immobilized to form a crosslinked probe complex. In some aspects, the immobilization comprises crosslinking the crosslinkable moiety of the immobilization oligonucleotide to the hybridized primary probe and attaching the attachment moiety to the biological sample or a matrix embedding the biological sample. In some aspects, the immobilization oligonucleotide is crosslinked to the primary probe, for example via the crosslinkable moiety. In some embodiments, the immobilization oligonucleotide is attached to the biological sample or a matrix embedding the biological sample, for example via the attachment moiety. In some embodiments, the crosslinkable moiety and attachment moiety may be any suitable crosslinkable moiety and attachment moiety, such as any described in Section III.

In some embodiments, immobilizing the immobilizable probe complex in the biological sample or matrix allows the immobilizable probe complex (i.e. crosslinked probe complex) to be detected at the position at which it is immobilized, including after subsequent processing steps, such as tissue clearing and/or enzymatic processing steps, such as digestion, e.g. of the target nucleic acid. In some aspects, the immobilizable probe complex is generated while contacting (e.g. hybridizing to, via the primary probe) the target nucleic acid. However, in some embodiments, once the immobilizable probe complex is immobilized to form the crosslinked probe complex, the target nucleic acid need not remain present and/or hybridized to the immobilizable probe complex in order to detect the presence and/or original position of the target nucleic acid in the biological sample or matrix. Thus, in some embodiments, the crosslinked probe complex can serve as an indicator of the presence and/or position of the target nucleic acid in the original biological sample, even after the target nucleic acid has been removed and/or de-hybridized from the crosslinked probe complex.

In some embodiments, a plurality of immobilization oligonucleotides are hybridized to the primary probe to form the immobilizable probe complex. In some embodiments, each immobilization oligonucleotide comprises a crosslinkable moiety. In some embodiments, each immobilization oligonucleotide comprises an attachment moiety.

In some embodiments, the immobilization oligonucleotide hybridizes to an overhang region of the primary probe (e.g. a 5′ or 3′ overhang region that does not hybridize to the target nucleic acid). In some embodiments, the immobilization oligonucleotide does not comprise a detectable label. Thus, in some embodiments, the immobilization oligonucleotide facilitates immobilization of the primary probe and immobilizable probe complex, but is not itself detected.

In some embodiments, the crosslinked probe complex can be detected at a position in the biological sample or matrix. In some embodiments, the detection is by any suitable method, such as any described herein in Section V. In some embodiments, the crosslinked probe complex is detected using a detection probe. In some embodiments, the detection probe comprises (i) a recognition sequence that hybridizes to a sequence of the overhang region of the primary probe, and (ii) a reporter sequence for binding directly or indirectly to a detectably labeled probe. In some embodiments, the detection probe is a first detection probe, and the method comprises removing the first detection probe after detecting the first detection probe, hybridizing a second detection probe to the overhang region, and detecting the second detection probe. Thus, in some embodiments, a sequence of signals can be generated, using different detection probes, wherein the sequence of signals corresponds to the target nucleic acid. In some embodiments, the detection probe is a circular or circularizable probe, and the method comprises performing rolling circle amplification of the detection probe and detecting a product thereof. In some embodiments, the detection probe is a detectably labeled probe.

In some embodiments, the method comprises hybridizing one or more secondary immobilization oligonucleotides to the immobilization oligonucleotide or plurality thereof. For example, the one or more secondary immobilization oligonucleotides can be hybridized to the immobilization oligonucleotide or plurality thereof to form a hybridization complex, such as a branched structure, for generating the crosslinked probe complex. In some embodiments, the one or more secondary immobilization oligonucleotide comprises an attachment moiety. In some embodiments, the method further comprises crosslinking the one or more secondary immobilization oligonucleotide to the biological sample or the matrix to form the crosslinked probe complex. In some embodiments, the crosslinkable moiety can be any suitable crosslinkable moiety, for example as described herein.

In some embodiments, the one or more immobilization oligonucleotide or plurality thereof comprises a universal hybridization region. In some embodiments, the hybridization region comprises (i) one or more universal or random bases and (ii) the crosslinkable moiety. In some embodiments, the universal hybridization region of the immobilization oligonucleotide or plurality thereof hybridizes non-specifically to the primary probe. For example, the universal hybridization region is capable of hybridizing to, and/or hybridizes to two or more different sequences in the primary probe.

### B. Probes or Probe Sets

In some embodiments, the methods provided herein comprise hybridizing a primary probe or probe set to a target sequence in the target nucleic acid. In some embodiments, the target sequence is specific to the target nucleic acid (e.g., the target sequence can identify the target nucleic acid). In some embodiments, the primary probe or probe set hybridizes to a target sequence in the target nucleic acid, and the immobilization oligonucleotide hybridizes to a hybridization region that is a common sequence (e.g., a polyA sequence) present in a plurality of different target nucleic acids. In some embodiments, the primary probe or probe set hybridizes to a specific target sequence in the target nucleic acid, and the immobilization oligonucleotide hybridizes non-specifically to the target nucleic acid (e.g., via a hybridization region comprising universal and/or random bases). In some embodiments, the primary probe or probe set and the immobilization oligonucleotide hybridizes to different sequences in the target nucleic acid.

In some cases, the primary probe or probe set is a barcoded probe or probe set. Examples of barcoded probes or probe sets may comprise a circularizable probe or probe set (e.g., based on a padlock probe, a gapped padlock probe, a SNAIL (Splint Nucleotide Assisted Intramolecular Ligation) probe set), a PLISH (Proximity Ligation in situ Hybridization) probe set, a RollFISH probe set, or a PLAYR (Proximity Ligation Assay for RNA) probe set). In some embodiments, the barcoded probe or probe set is not circular or circularizable. Examples of barcoded probes or probe sets include, but are not limited to, L-shaped probes (e.g., a probe comprising a target-hybridizing sequence and a 5′ or 3′ overhang upon hybridization to its target sequence), or U-shaped probes (e.g., a probe comprising a target-hybridizing sequence and a 5′ overhang and a 3′ overhang upon hybridization to its target sequence). The specific probe or probe set design can vary.

In some embodiments, the primary probe or probe set is a probe comprising a 3′ or 5′ overhang upon hybridization to the target nucleic acid (e.g., an L-shaped probe, as shown in FIG. 2A). In some embodiments, the primary probe or probe set is a probe comprising a 3′ overhang and a 5′ overhang upon hybridization to the target nucleic acid (a U-shaped probe). In some embodiments, the 3′ overhang and the 5′ overhang each independently comprises one or more detectable labels and/or barcode sequences. In some embodiments, the 3′ and/or 5′ overhang comprises one or more detectable labels and/or barcode sequences. In some embodiments, multiple primary probes comprising one or more overhang regions are hybridized to a plurality of target sequences within a particular target nucleic acid molecule (e.g., tiling across multiple regions in the target nucleic acid molecule). Such tiling of probes can provide signal amplification by increasing the number of detectable labels and/or barcode sequences per target nucleic acid. For example, between 10 and 20, between 10 and 30, or between 20 and 40 probes can be hybridized per target nucleic acid molecule. In some embodiments according to the methods described herein, each target nucleic acid molecule can be hybridized by no more than 1, 2, 3, 4, 5, 6, 7, 8, 9, or 10 primary probes. In some embodiments, a single probe is hybridized per target nucleic acid molecule (e.g., per target RNA). Any suitable method of signal amplification can be used to detect a barcode sequence in the overhang region of the primary probe (e.g., RCA of a probe that directly or indirectly binds to the primary probe or probe set and/or the amplification product thereof; hybridization chain reaction (HCR) directly or indirectly on the primary probe or probe set and/or the amplification product thereof; linear oligonucleotide hybridization chain reaction (LO-HCR) directly or indirectly on the primary probe or probe set and/or the amplification product thereof; primer exchange reaction (PER) directly or indirectly on the primary probe or probe set and/or the amplification product thereof; assembly of branched structures directly or indirectly on the primary probe or probe set and/or the amplification product thereof; hybridization of a plurality of detectable probes directly or indirectly on the primary probe or probe set and/or the amplification product thereof, or any combination thereof).

In some embodiments, the primary probe or probe set is a circular probe. In some embodiments, the primary probe or probe set is a circularizable probe or probe set. In some embodiments, the primary probe or probe set is designed for RNA-templated ligation. See, e.g., U.S. Pat. Pub. 2020/0224244 which is hereby incorporated by reference in its entirety. In any of the embodiments herein, the circularizable probe or probe set can comprise one, two, three, four, or more ribonucleotides. In some embodiments, the circularizable probe or probe set is designed to be circularized using the target nucleic acid (e.g., a DNA or RNA target nucleic acid) as a template. In some embodiments, the circularizable probe or probe set is designed to be circularized using another probe as a template (e.g., as in the case of SNAIL or RollFISH probes). In some embodiments, the probe used as a template for circularization is also used as a primer for amplification of the circularized probe or probe set. In some embodiments, a separate primer is provided for amplification of the circularized probe or probe set. Any other modifications or variations of circularizable probe or probe sets can be used.

In some embodiments, the primary probe or probe set comprises a primer binding site. In some embodiments, a primer is provided for hybridization to the primer binding site, wherein the primer can be extended to form an amplification product of the probe or probe set (e.g., a rolling circle amplification product of a circular or circularized probe). A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the primary probe or probe set comprises a first probe and a second probe that can be ligated to generate a ligated first-second probe (e.g., a linear ligated probe). In some embodiments, a linear ligated probe can be circularized using an additional bridge probe that is ligated to either end of the ligated linear probe (e.g., in a templated or non-templated ligation). In some embodiments, the first and/or second probe comprises an overhang region, which may optionally comprise one or more barcode sequences for detection of the first and/or second probe or the ligated first-second probe. In some embodiments, the probe or probe set is a circularizable probe or probe set (e.g., a padlock probe). In some embodiments, the circularizable probe or probe set comprises one or more barcode sequences for detection of circularizable probe or probe set, the circularized probe or probe set, or an amplification product thereof.

In some embodiments, a primary probe or probe set disclosed herein can comprise one, two, three, four, or more ribonucleotides in a DNA backbone. In any of the embodiments herein, the one or more ribonucleotides can be at and/or near a ligatable 3′ end of a circularizable probe or probe set. The probe or probe may comprise an optional 3′ RNA base. In some embodiments, a probe or probe set disclosed herein can comprise a 5′ flap which may be recognized by a structure-specific cleavage enzyme (e.g. an enzyme capable of recognizing the junction between single-stranded 5′ overhang and a DNA duplex and cleaving the single-stranded overhang). In some embodiments, the flap is positioned between a 3′ end and 5′ end of a split hybridization region upon hybridization of the primary probe or probe set to the target sequence, and cleavage of the flap allows ligation of the 3′ end to the 5′ end of the split hybridization region. Methods of ligating a first and second hybridization region with or without flap cleavage are described in U.S. Pat. Pub. 20200224244, the entire content of which is herein incorporated by reference.

In some embodiments, the primary probe or probe set comprises a split hybridization region configured to hybridize to a splint. In some embodiments, the split hybridization region comprises one or more barcode sequences. For example, a probe set can comprise two probes that hybridize to adjacent portions of the target sequence, wherein each probe comprises an overhang region that does not hybridize to the target nucleic acid. The overhang regions can together form a split-hybridization region, either in a double “Z”-like configuration or a double “U”-like configuration. The split hybridization region can comprise one or more barcode sequences specific to the target sequence, so that the target sequence can be identified by hybridizing a detectable splint to the split hybridization region. The splint may be directly or indirectly labeled. In some embodiments, the splint is a bridge probe. In some embodiments, the splint is ligated to one or more other probes (e.g., to form a circularized probe), and optionally amplified by rolling circle amplification, In some embodiments, the splint comprises a barcode sequence (e.g., in an overhang region) that can be detected using any of the signal amplification and detection methods described herein, such as assembly of branched DNA structures, HCR, LO-HCR, RCA, PER, etc. Examples of probes or probe sets comprising split hybridization regions (e.g., Z-probes, proximity ligation in situ hybridization (PLISH) probes, or split-FISH probes) have been described, for example, in U.S. Pat. Pub. 20160115555, U.S. Pat. Pub. US20200224243, U.S. Pat. Pub. 20160108458, and WO2021/167526, the contents of each of which are herein incorporated by reference in their entireties.

In some embodiments described herein, the primary probe or probe set comprises a target recognition region (optionally a split target recognition region) capable of hybridizing to a target sequence in or associated with an analyte in a biological sample. In some embodiments, the target recognition region is complementary to the target sequence. In some embodiments, the target recognition region is at least about 10, at least about 15, at least about 20, at least about 25, at least about 30, at least about 35, at least about 40, at least about 45, at least about 50, at least about 70, at least about 80, at least about 90, or at least about 100 nucleotides in length. In some embodiments, the target recognition region is between or between about any one of 5 and 200, 10 and 200, 15 and 200, 20 and 200, 5 and 100, 10 and 100, 15 and 100, 20 and 100, 5 and 50, 10 and 50, 15 and 50, 20 and 50, 5 and 20, or 10 and 40 nucleotides in length. In some embodiments, the target recognition region is a split target recognition region.

In some embodiments, the split target recognition region comprises a first target recognition region and a second target recognition region. In some embodiments, the first target recognition region is at a first end of a probe or probe set and the second target recognition region is at a second end of a probe or probe set. In some embodiments, the first target recognition region is at a 3′ end of a probe or probe set and the second target recognition region is at a 5′ end of a probe or probe set, or vice versa. In some embodiments, the first and second target recognition regions are at a first and second end of a circularizable probe. In some embodiments, the first and second target recognition regions are in a first and second probe. In some embodiments, the 3′ or 5′ end of the probe or probe set comprises a flap (e.g., an overhang region that does not hybridize to the target nucleic acid) that is cleaved prior to ligation of the probe or probe set. In some embodiments, the first target recognition region and the second target recognition region are independently between or between about any one of 5 and 200, 10 and 200, 15 and 200, 20 and 200, 5 and 100, 10 and 100, 15 and 100, 20 and 100, 5 and 50, 10 and 50, 15 and 50, 20 and 50, 5 and 20, or 10 and 40 nucleotides in length.

In some embodiments, the probe or probe set comprises an anchor sequence, which can be a common sequence among a plurality of probes or probe sets for a plurality of target sequences. In some embodiments, the method comprises contacting the sample with an anchor probe configured to hybridize to the anchor sequence or a complement thereof. In some embodiments, the anchor probe is complementary to the anchor sequence or complement thereof. In some embodiments, the anchor probe is a detectable probe. The anchor probe can be directly labeled or indirectly labeled (e.g., by direct or indirect hybridization of one or more detectably labeled probes to the anchor probe). In some embodiments, the method comprises imaging the sample to detect hybridization of the anchor probe, thereby detecting a plurality of analytes simultaneously.

In some embodiments, the target sequence is a marker sequence for a particular analyte, which identifies the particular analyte (e.g., alone or in combination with one or more other marker sequences). Thus, in some embodiments, a target sequence for a given target analyte is specific to that analyte, or unique, such that multiple target analytes can be distinguished from each other. In some embodiments, the analyte is an RNA molecule (e.g., an endogenous RNA molecule). Various analytes that may comprise target sequences, and methods of associating target sequences with different analytes, are described in Section VI.B below.

In some embodiments, the target sequence is present in a group of related molecules, e.g. isoforms or variants or mutants of an RNA transcript for a given gene. In some embodiments, the target sequence is specific to a particular subset of molecules (e.g., specific to a particular variant or mutant of an endogenous analyte such as an RNA molecule. For example, in some embodiments, the target sequence comprises a particular single nucleotide variant. In some embodiments, the target sequence may be unique or specific to the particular variant. In this way different variants, or isoforms, or mutants may be identified or distinguished from one another using the primary probes or probe sets.

Where the analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte or labeling agent as a tag or identifier (ID) sequence (e.g. a barcode) for the analyte or labeling agent. It may thus be a synthetic or artificial sequence.

In some embodiments, the probe or probe set comprises one or more barcode sequences or complements thereof. The barcode sequences may be positioned anywhere within the nucleic acid probe or probe set. If more than one barcode sequence is present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart. In some embodiments, one or more barcodes are indicative of the target sequence in the target nucleic acid, such as a single nucleotide of interest (e.g., SNPs or point mutations), a dinucleotide sequence, or a short sequence of about 5 nucleotides in length in the target sequence.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

### C. Primary Immobilizable Probes

In some aspects, provided herein is a method of analyzing a biological sample, comprising performing an extension reaction of a primary probe hybridized to a target nucleic acid in the biological sample to incorporate an attachment moiety using the target nucleic acid as a template. In some embodiments, the attachment moiety is a crosslinkable nucleotide, and the incorporation of the attachment moiety leads to the formation of a primary immobilizable probe. In some embodiments, the method comprises crosslinking the primary immobilizable probe in the biological sample or a matrix embedding the biological sample. In some embodiments, the method comprises detecting the crosslinked primary immobilizable probe at a position in the biological sample or matrix.

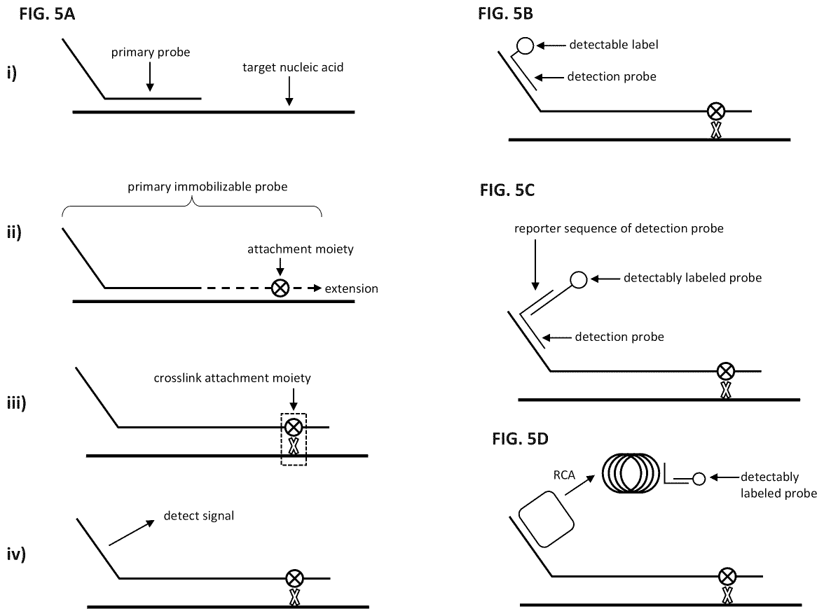

In some aspects, provided herein are immobilizable probes, such as a primary immobilizable probe. In some aspects, the primary immobilizable probe is a primary probe that hybridizes to a target nucleic acid, and comprises an attachment moiety for immobilization. In some embodiments, the primary immobilizable probe is generated from a primary probe that is not immobilizable. In some embodiments, the primary probe hybridizes to the target nucleic acid, and the primary probe is extended in an extension reaction (e.g., using a polymerase) to incorporate an attachment moiety using the target nucleic acid as template, thereby generating the primary immobilizable probe that is hybridized to the target nucleic acid. FIG. 5A provides a schematic illustration of a workflow wherein a primary probe is (i) hybridized to a target nucleic acid, and (ii) extended using a polymerase to incorporate one or more nucleotides functionalized with an attachment moiety using the target nucleic acid as a template, thereby forming a primary immobilizable probe. The attachment moiety can then be attached to the biological sample and/or to a matrix embedding the biological sample. In some embodiments, the one or more nucleotides functionalized with an attachment moiety comprise one or more crosslinkable groups for interstrand crosslinking to the target nucleic acid. In some embodiments, the one or more nucleotides functionalized with an attachment moiety comprise one or more nucleotides functionalized with an amine, a thiol, an azide, an alkyne, a nitrone, an alkene, a tetrazine, a tetrazole, an acrydite, or any click reactive group not listed above.

In some embodiments, the method comprises detecting the primary immobilizable probe at a position in the biological sample or matrix (e.g., after attaching the primary immobilizable probe to the biological sample or matrix. In some embodiments, the primary immobilizable probe is detectably labeled (e.g., with a fluorescent dye). In some embodiments, the primary immobilizable probe comprises an overhang region that does not hybridize to the target nucleic acid, and the method comprises hybridizing a detection probe to the overhang region and detecting the detection probe or a product thereof. In some embodiments, the detection probe comprises a recognition sequence that hybridizes to a sequence of the overhang region and a detectable label, as illustrated in FIG. 5B. In some embodiments, the detection probe comprises a recognition sequence that hybridizes to a sequence of the overhang region and a reporter sequence for binding directly or indirectly to a detectably labeled probe, as illustrated in FIG. 5C. In some embodiments, the detection probe is a first detection probe, and the method comprises removing the first detection probe after detecting the first detection probe, hybridizing a second detection probe to the overhang region, and detecting the second detection probe (e.g., by detecting a label such as a fluorescent dye that is attached to the detection probe or to a detectably labeled probe that binds directly or indirectly to the detection probe). Thus, in some embodiments, a sequence of signals (a series of signals, also referred to as a signal code sequence) can be generated, using different detection probes, wherein the sequence of signals corresponds to the target nucleic acid. In some embodiments, the detection probe is a circular or circularizable probe, and the method comprises performing rolling circle amplification of the detection probe and detecting a product thereof, as illustrated in FIG. 5D. In some embodiments, RCA is performed using the immobilizable probe as a primer and the circular or circularized probe (e.g., detection probe) as template. In some embodiments, RCA is performed using a separate primer and the circular or circularized probe (e.g., detection probe) as template. In some embodiments, the detection probe is a circularizable probe or probe set, and the method comprises circularizing the circularizable probe or probe set to form a circularized probe, and performing rolling circle amplification of the circularized probe. In certain embodiments, the circularizable probe or probe set is circularized using the target nucleic acid and/or a splint oligonucleotide as template(s) for ligation. It will be understood that the rolling circle amplification product can be detected by direct binding of detectably labeled probes to the rolling circle amplification product or indirect binding of detectably labeled probes to the rolling circle amplification product, as shown in FIG. 5D.

In some embodiments, the primary immobilizable probe is crosslinked in the biological sample or a matrix embedding the biological sample. In some embodiments, the primary immobilizable probe is crosslinked using the attachment moiety. In some embodiments, the incorporated attachment moiety may be any suitable attachment moiety as described herein, such as a crosslinkable nucleotide, or any other crosslinkable moiety as described in Section III. In some embodiments, crosslinking the primary immobilizable probe in the biological sample or matrix allows the crosslinked primary immobilizable probe to be detected at the position at which it is immobilized, including after subsequent processing steps, such as tissue clearing and/or enzymatic processing steps, such as digestion, e.g. of the target nucleic acid. In some aspects, the primary immobilizable probe is generated using the target nucleic acid as a template, and thus is hybridized to the target nucleic acid while it is generated. However, in some embodiments, once the primary immobilizable probe is crosslinked in the biological sample or matrix, the target nucleic acid need not remain present and/or hybridized to the crosslinked primary immobilizable probe in order to detect the presence and/or original position of the target nucleic acid in the biological sample or matrix. Thus, in some embodiments, the primary immobilizable probe can serve as an indicator of the presence and/or position of the target nucleic acid in the original biological sample, even after the target nucleic acid has been removed and/or de-hybridized from the primary immobilizable probe.

In some embodiments, the primary immobilizable probe can be detected at a position in the biological sample or matrix. In some embodiments, the primary immobilizable probe can be detected by any suitable means, such as any described herein in Section V. In some embodiments, the primary immobilizable probe comprises an overhang region, such as at its 5′ end. In some embodiments, the overhang is originally comprised by the primary probe, and thus is also comprised in the extended primary immobilizable probe. In some embodiments, the 5′ overhang of the primary immobilizable probe facilitates detection of the primary immobilizable probe by any suitable means for detection. For example, the overhang may comprise one or more sequences, such as sequences of barcode regions, that can be detected, e.g., using secondary probes, higher order probes, and/or detectably labeled oligonucleotides, such as any described herein. In some embodiments, the 5′ overhang is detected using a method involving an amplification reaction, such as rolling circle amplification of a circular or circularized template hybridized thereto.

In some embodiments, the method comprises hybridizing a detection probe to the overhang region and detecting the detection probe or a product thereof. In some embodiments, the detection probe comprises (i) a recognition sequence that hybridizes to a sequence of the overhang region. In some embodiments, the detection probe further comprises (ii) a reporter sequence for binding directly or indirectly to a detectably labeled probe. In some embodiments, the detection probe is a first detection probe, and the method comprises removing the first detection probe after detecting the first detection probe, hybridizing a second detection probe to the overhang region, and detecting the second detection probe. Thus, in some embodiments, a sequence of signals can be generated, using different detection probes, wherein the sequence of signals corresponds to the target nucleic acid. In some embodiments, the detection probe is a circular or circularizable probe, and the method comprises performing rolling circle amplification of the detection probe and detecting a product thereof. In some embodiments, the detection probe is a detectably labeled probe.

In some embodiments, a plurality of primary immobilizable probes can be generated. In some embodiments, the method comprises performing extension reactions of a plurality of primary probes hybridized to the target nucleic acid, for example at different sequences within the same target nucleic acid. In some embodiments, the extension reactions incorporate attachment moieties into the primary probes, thereby generating the plurality of primary immobilizable probes. In some embodiments, each primary probe of the plurality of primary probes (and thus each primary immobilizable probe) comprises an overhang region at its 5′ end, e.g. for detection, as described above. In some embodiments, the extension reactions are performed simultaneously. In some embodiments, the plurality of primary immobilizable probes can be detected, e.g. by any of the detection methods described above. In some embodiments, a signal, or a combination of signals, such as a sequence of signals, can be generated from the plurality of primary immobilizable probes and detected in order to identify the target nucleic acid.

In some embodiments, a polymerase for the extension reaction performed using the primary probe as a primer lacks strand displacing activity. In some embodiments, a polymerase lacking strand displacement activity allows simultaneous extension reactions to be performed to extend a plurality of primary probes hybridized to the same target nucleic acid, without the extension of one primary probe leading to the displacement of another primary probe hybridized to the target nucleic acid. Thus, in some embodiments, the extension reaction of a primary probe of the plurality of primary probes does not displace other primary probes of the plurality of primary probes from the target nucleic acid. In some embodiments, the extension reaction or simultaneous extension reactions can be performed for a duration of time, such as less than 30 minutes, less than 10 minutes, or less than 5 minutes. In some embodiments, performing an extension reaction for a limited duration of time can reduce the probability of primary probe displacement, for example in instances where the polymerase exhibits strand displacement activity.

In some embodiments, a primary immobilizable probe is generated in the biological sample. In some embodiments, a method provided herein comprises contacting the biological sample with a primary probe that hybridizes to a target nucleic acid in the biological sample, and ligating the primary probe to an oligonucleotide comprising any of the attachment moieties and/or crosslinkable moieties disclosed herein to generate the primary immobilizable probe. In some embodiments, the primary probe is ligated to the oligonucleotide comprising the attachment moiety and/or crosslinkable moiety using the target nucleic acid as a template. In some embodiments, the primary probe is ligated to the oligonucleotide comprising the attachment moiety and/or crosslinkable moiety using a splint that hybridizes to at least a portion of the primary probe and at least a portion of the oligonucleotide comprising the attachment moiety and/or crosslinkable moiety. In some embodiments, the splint is between 10 nucleotides and 200 nucleotides in length (e.g., between 20 and 100, between 20 and 80, between 20 and 60 nucleotides, or between 10 and 60 nucleotides in length). In some embodiments, the portion of the oligonucleotide that hybridizes to the splint is between about 5 and 100 nucleotides in length (e.g., between 5 and 60, between 5 and 50, between 10 and 40, between 10 and 30, between 10 and 25, or between 5 and 25 nucleotides in length). In some embodiments, the portion of the primary probe that hybridizes to the splint is between about 5 and 100 nucleotides in length (e.g., between 5 and 60, between 5 and 50, between 10 and 40, between 10 and 30, between 10 and 25, or between 5 and 25 nucleotides in length).

In some aspects, provided herein is a method of analyzing a biological sample, comprising: (a) contacting the biological sample with an immobilization oligonucleotide and a primary probe, wherein the primary probe hybridizes to a target nucleic acid in the biological sample, and wherein the immobilization oligonucleotide comprises an attachment moiety; (b) ligating the primary probe to the immobilization oligonucleotide to form a ligated immobilizable probe comprising the primary probe and the immobilization oligonucleotide; (c) crosslinking the attachment moiety of the ligated immobilizable probe to a matrix embedding the biological sample, thereby crosslinking the immobilizable probe to the matrix; (d) contacting the biological sample with a detection probe that hybridizes to the ligated immobilizable probe or a product thereof; and (e) detecting the detection probe or a product of the detection probe at a position in the biological sample. In some embodiments, the detection probe hybridizes to a detection probe hybridization sequence in a first overhang region of the primary probe in the immobilizable probe. In some embodiments, the method comprises contacting the immobilization oligonucleotide with a splint that hybridizes to at least a portion of the primary probe and at least a portion of the immobilization oligonucleotide. In some embodiments, the splint is between 10 nucleotides and 200 nucleotides in length (e.g., between 20 and 100, between 20 and 80, between 20 and 60 nucleotides, or between 10 and 60 nucleotides in length). In some embodiments, the portion of the immobilization oligonucleotide that hybridizes to the splint is between about 5 and 100 nucleotides in length (e.g., between 5 and 60, between 5 and 50, between 10 and 40, between 10 and 30, between 10 and 25, or between 5 and 25 nucleotides in length). In some embodiments, the portion of the primary probe that hybridizes to the splint is between about 5 and 100 nucleotides in length (e.g., between 5 and 60, between 5 and 50, between 10 and 40, between 10 and 30, between 10 and 25, or between 5 and 25 nucleotides in length). In some embodiments, the splint serves as a template for ligating the primary probe to the immobilization oligonucleotide. In some embodiments, the portion of the primary probe that hybridizes to a splint is a second overhang region of the primary probe. In some embodiments, the primary probe comprises the first overhang region at the 3′ end of the primary probe and the second overhang region at the 5′ end of the primary probe. In some embodiments, the primary probe comprises the first overhang region at the 5′ end of the primary probe and the second overhang region at the 3′ end of the primary probe. In some embodiments, the immobilization oligonucleotide and the primary probe hybridize to adjacent sequences of the target nucleic acid. In some embodiments, the target nucleic acid serves as a template for ligating the primary probe to the immobilization oligonucleotide. In some embodiments, the primary probe and/or the immobilization oligonucleotide further comprise a crosslinkable moiety for interstrand crosslinking and/or a second attachment moiety. The crosslinkable moiety and/or second attachment moiety can be any of the crosslinkable moieties or attachment moieties described herein.

### D. Secondary and Tertiary Immobilizable Probes

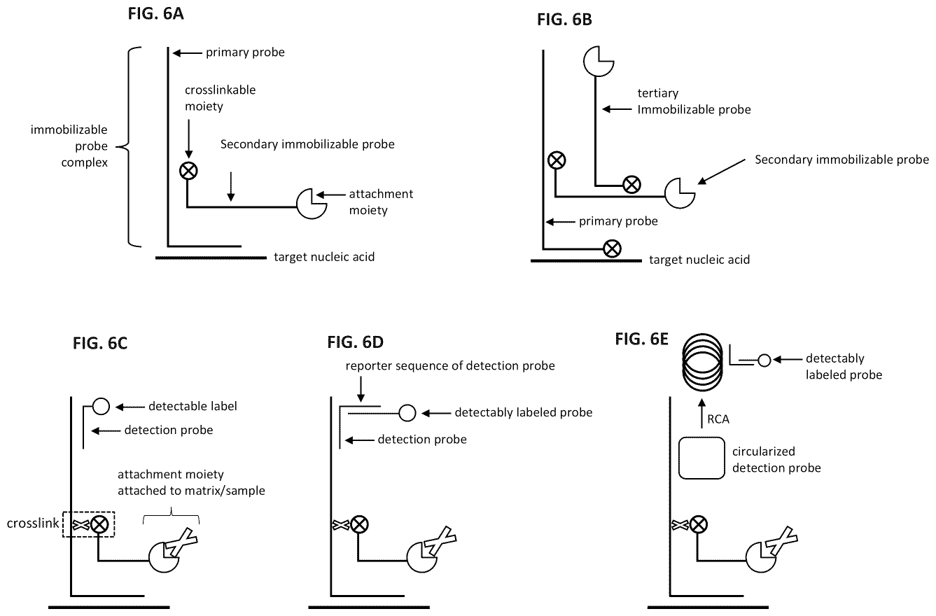

In some embodiments, provided herein are secondary immobilizable probes that hybridize to primary probes in a biological sample. In some embodiments, provided herein are tertiary immobilizable probes that hybridize to secondary probes, which in turn hybridize to primary probes in the biological sample. In some embodiments, an immobilizable probe complex is formed comprising a primary probe and a secondary immobilizable probe hybridized to the primary probe. In some cases, an interstrand crosslink is formed between the primary probe and the secondary immobilizable probe. In some embodiments, an immobilizable probe complex is formed between a primary probe, a secondary immobilizable probe, and a tertiary immobilizable probe. In some embodiments, an interstrand crosslink is formed between the primary probe and the secondary immobilizable probe, and an interstrand crosslink is formed between the secondary immobilizable probe and the tertiary immobilizable probe. In some embodiments, an interstrand crosslink is formed between the primary probe and the target nucleic acid in the biological sample. In some embodiments, the target nucleic acid is an RNA, such as an mRNA. In some embodiments, the target nucleic acid is a DNA, such as a cDNA or an oligonucleotide in a labeling agent (e.g., an oligonucleotide conjugated to an antibody). As shown in FIG. 6A, in some embodiments, the secondary immobilizable probe is functionalized with a crosslinkable moiety for interstrand crosslinking, and an attachment moiety for attachment to the biological sample or to a matrix embedding the biological sample. Any of the crosslinkable moieties or attachment moieties described herein can be used. As shown in FIG. 6B, in some embodiments, the tertiary immobilizable probe is functionalized with a crosslinkable moiety for interstrand crosslinking, and an attachment moiety for attachment to the biological sample or to a matrix embedding the biological sample. Any of the crosslinkable moieties or attachment moieties described herein can be used. Also as shown in FIG. 6B, in some embodiments, the primary probe comprises a crosslinkable moiety for interstrand crosslinking.

In some embodiments, provided herein is a method of analyzing a biological sample, comprising: (a) contacting the biological sample with a primary probe; (b) hybridizing a secondary immobilizable probe to the primary probe to form an immobilizable probe complex, wherein the secondary immobilizable probe is functionalized with a crosslinkable moiety and an attachment moiety, (c) crosslinking the crosslinkable moiety of the immobilization oligonucleotide to the hybridized primary probe and attaching the attachment moiety to the biological sample or a matrix embedding the biological sample, thereby forming a crosslinked probe complex, and (d) hybridizing a detection probe to the primary probe and detecting the detection probe or a product thereof, thereby detecting the crosslinked probe complex at a position in the biological sample or matrix. In some embodiments, the method comprises hybridizing a plurality of secondary immobilizable probes to the primary probe to form the immobilizable probe complex, wherein each secondary immobilizable probe comprises an attachment moiety, and wherein the method comprises, using the attachment moiety, attaching each secondary immobilizable probe to the biological sample or the matrix to form the crosslinked probe complex. In some embodiments, the secondary immobilizable probe or plurality thereof hybridizes to an overhang region of the primary probe (a region that is not hybridized to the target nucleic acid). In some embodiments, the secondary immobilizable probe or plurality thereof does not comprise a detectable label. In some embodiments, the tertiary immobilizable probe or plurality thereof does not comprise a detectable label. In some embodiments, the secondary and tertiary immobilizable probes or plurality thereof do not comprise a detectable label. In some embodiments, the method comprises hybridizing a tertiary immobilizable probe comprising an attachment moiety to the secondary probe or plurality thereof. In some embodiments, the method comprises crosslinking the tertiary immobilizable probe to the biological sample or the matrix to form the crosslinked probe complex.

In any of the above embodiments, a detection probe can be used to detect the primary probe in the biological sample. In some embodiments, the detection probe is a detectably labeled probe, as illustrated in FIG. 6C. In some embodiments, the detection probe comprises (i) a recognition sequence that hybridizes to a sequence of the overhang region of the primary probe, and (ii) a reporter sequence for binding directly or indirectly to a detectably labeled probe, as illustrated in FIG. 6D. In some embodiments, the detection probe is a first detection probe, and the method comprises removing the first detection probe after detecting the first detection probe, hybridizing a second detection probe to the overhang region, and detecting the second detection probe. In some embodiments, the detection probe is a circular or circularizable probe, and the method comprises performing rolling circle amplification of the detection probe and detecting a the rolling circle amplification product of the circularized detection probe (e.g., using detectably labeled probe that binds directly to the RCP, or a detectably labeled probe that binds indirectly to the RCP via an intermediate probe, as shown in FIG. 6E).

### E. Gap-Fill Incorporation of a Crosslinkable Nucleotide in a Hybridized Probe or Probe Set

In some embodiments, provided herein is a method of analyzing a biological sample, comprising: (a) contacting the biological sample with a probe or probe set comprising a first hybridization region and a second hybridization region, wherein the first hybridization region and the second hybridization region hybridize to a first and second target sequence, respectively, in a target nucleic acid, wherein the first and second target sequences are 3′ and 5′, respectively, to a first sequence of a region of interest in the target nucleic acid, wherein the region of interest comprises a first nucleobase; (b) contacting the biological sample with a crosslinkable nucleotide complementary to the first nucleobase; (c) extending the 3′ end of the first hybridization region with a polymerase using the first sequence of the region of interest as a template, thereby incorporating the crosslinkable nucleotide into the first hybridization region; (d) ligating the extended 3′ end of the first hybridization region and the 5′ end of the second hybridization region to form a ligated probe; (e) crosslinking the incorporated crosslinkable nucleotide to the biological sample or a matrix embedding the biological sample; and (f) detecting the crosslinked ligated probe or a product thereof. In some embodiments, the crosslinked ligated probe or a product thereof is detected at a location in the biological sample or matrix. In some embodiments, the target nucleic acid is DNA. In some embodiments, the target nucleic acid is a cDNA.

In some embodiments, an alternative sequence of the region of interest does not comprise the first nucleobase, such that the crosslinkable nucleotide is not incorporated into the circularizable probe when using the alternative sequence of the region of interest as a template. For example, the method could be used to distinguish between a first sequence of a region of interest in the target nucleic acid and one or more alternative sequences of the region of interest. The first sequence of the region of interest comprises a particular nucleobase (e.g., an A, T, C, or G in a DNA region of interest, or an A, U, C, or G in an RNA region of interest). The alternative sequences of the region of interest do not comprise the particular nucleobase. A probe or probe set is designed to hybridize to sequences flanking the region of interest in the target nucleic acid. The method can comprise contacting the biological sample with a polymerase and a nucleotide mixture for extension of the 3′ end of the first hybridization region hybridized to a sequences 3′ to the region of interest in the target nucleic acid, such that extending the 3′ end of the first hybridization region uses the region of interest as a template. The nucleotide mixture can comprise a crosslinkable nucleotide that is complementary to the particular nucleobase present in the first sequence of the region of interest, but absent in the one or more alternative sequences of the region of interest. If the first sequence of interest comprising the particular nucleobase is present at the region of interest, the crosslinkable nucleotide is incorporated into the hybridized probe. If particular nucleobase is not present in the region of interest, the crosslinkable nucleotide is not incorporated when the extended 3′ end of the first hybridization region is ligated to the 5′ end of the second hybridization region to form a ligated probe. The method can comprise performing one or more stringent washes to remove non-crosslinked probes from the biological sample. After removing the non-crosslinked probes from the biological sample, the method can comprise detecting the crosslinked ligated probe, thereby detecting the first sequence of the region of interest. In some embodiments, the target nucleic acid is DNA. In some embodiments, the target nucleic acid is a cDNA.

In some embodiments, the ligatable probe or probe set is a ligatable probe set comprising a first part and a second part, wherein the first part comprises the first hybridization region and the second part comprises the second hybridization region. In some cases, upon ligation, the first part and second part form a linear ligated probe. In some embodiments, the first part and/or the second part comprise an overhang region that does not hybridize to the target nucleic acid. For example, the first part can comprise a 5′ overhang region, and/or the second part can comprise a 3′ overhang region. In some embodiments, the one or more overhang regions comprise one or more barcode sequences corresponding to the first sequence of the region of interest. In some embodiments, detecting the crosslinked ligated probe comprises hybridizing a probe or probe set to a barcode sequence in the crosslinked ligated probe, and detecting the probe or probe set. For example, detecting the crosslinked ligated probe can comprise hybridizing a circularizable probe or probe set to an overhang region in the crosslinked ligated probe, circularizing the probe or probe set by one or more ligations to form a circularized probe, amplifying the circularized probe to generate a rolling circle amplification product (RCP), and detecting the RCP). A crosslinked ligated probe can be detected using detection and analysis methods described in Section VI.

In some embodiments, the ligatable probe or probe set is a circularizable probe or probe set. In some embodiments, provided herein is a method of analyzing a biological sample, comprising: (a) contacting the biological sample with a circularizable probe comprising (i) a 3′ arm that hybridizes to a first target sequence in a target nucleic acid in the biological sample, and (ii) a 5′ arm that hybridizes to a second target sequence in the target nucleic acid, wherein the first and second target sequence are 3′ and 5′, respectively, to a first sequence of a region of interest comprising a first nucleobase; (b) contacting the biological sample with a crosslinkable nucleotide complementary to the first nucleobase; (c) extending the 3′ arm of the circularizable probe with a polymerase using the first sequence of the region of interest as a template, thereby incorporating the crosslinkable nucleotide into the circularizable probe; (d) ligating the extended 3′ arm and the 5′ arm of the circularizable probe to form a circularized probe; (e) crosslinking the incorporated crosslinkable nucleotide to the biological sample or to a matrix embedding the biological sample; and (f) detecting the crosslinked circularized probe or a product thereof at a location in the biological sample or matrix. In some embodiments, an alternative sequence of the region of interest does not comprise the first nucleobase, such that the crosslinkable nucleotide is not incorporated into the circularizable probe when using the alternative sequence of the region of interest as a template. In some embodiments, the target nucleic acid is DNA. In some embodiments, the target nucleic acid is a cDNA.

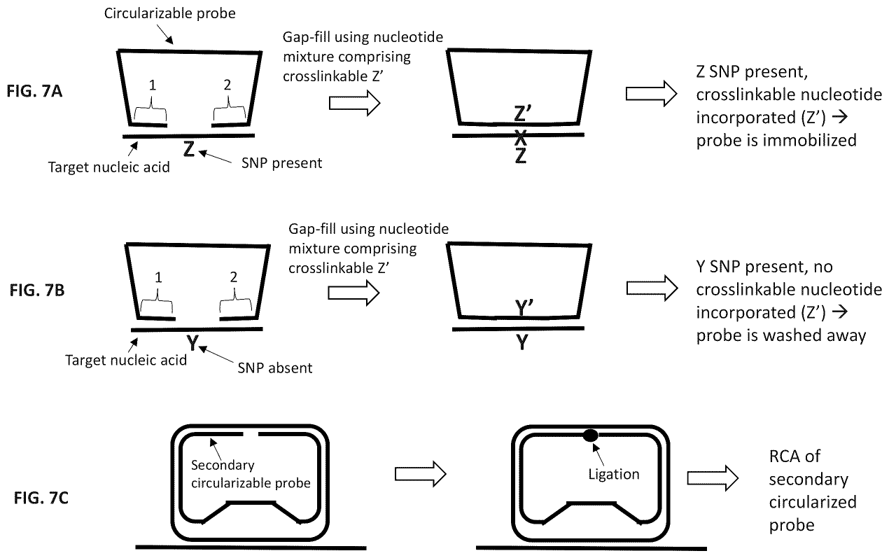

In some embodiments, detecting the crosslinked circularized probe or a product thereof comprises performing rolling circle amplification (RCA) using the circularized probe as a template to form a rolling circle amplification product (RCP) and detecting the RCP in the sample. In some embodiments, the method comprises decrosslinking the circularized probe prior to performing RCA. In some cases, the method comprises (a) hybridizing a secondary circular probe to the crosslinked circularized probe, or hybridizing a secondary circularizable probe to the crosslinked circularized probe and circularizing the hybridized secondary circularizable probe to generate a secondary circularized probe, and (b) performing RCA using the secondary circular probe or secondary circularized probe as a template to form a rolling circle amplification product (RCP) and detecting the RCP in the sample. In some embodiments, the method comprises detecting an RCP product formed from the secondary circularizable probe, and the method does not comprise decrosslinking the first circularized probe prior to RCA. FIG. 7C illustrates an embodiment comprising hybridizing a secondary circularizable probe to a crosslinked circularized probe, ligating the secondary circularizable probe to form a secondary circularized probe, and performing RCA of the secondary circularized probe. In some embodiments, the RCA product of the secondary circularized probe is detected in order to detect the crosslinked circularized probe. For example, a barcode sequence in the RCA product is detected and associated with the region of interest (e.g., SNP).

The following example is provided for illustration purposes only, as an example for detecting a single nucleotide polymorphism (SNP) at a region of interest in a target nucleic acid, that is either a C or a G. In this example, a signal would be detected only when there is a C. The first sequence of the region of interest is ATTCGTA, and the alternative variant sequence of the region of interest is ATTGGTA. A biological sample comprising the target nucleic acid is contacted with a probe or probe set comprising a first hybridization region and a second hybridization region, wherein the first hybridization region and the second hybridization region hybridize to a first and second target sequence, respectively, in a target nucleic acid, wherein the first and second target sequences are 3′ and 5′, respectively, to the region of interest. The first hybridization can be extended and ligated to the second hybridization using a polymerase and a ligase for gap-fill and ligation. In order to selectively crosslink the gap-fill ligated probe to the target nucleic acid when the SNP of interest (C) is present, the gap-fill extension reaction can be performed with only one crosslinkable nucleotide in the extension mix, which is a crosslinkable G. In some embodiments, in the extension mix, the nucleotide complementary to the SNP comprises a crosslinkable moiety and the nucleotide(s) complementary to other alternative variants do not comprise a crosslinkable moiety. Importantly, the alternative sequence of the region of interest does not comprise any of the nucleobase of interest (C). In this way, only when there is the C at the SNP site, the crosslinkable G is incorporated by the gap-fill extension. Thus, the ligated probe is only crosslinked to the target nucleic acid when the SNP of interest is present. The crosslinked ligated probe remains immobilized while non-crosslinked probes are removed using one or more stringent washes.

FIGS. 7A-7B depict an example of a workflow comprising gap-fill incorporation of a crosslinkable nucleotide (depicted as Z′) complementary to a SNP (depicted as Z) in a region of interest to detect the SNP in a target nucleic acid. FIG. 7A illustrates an example where the SNP (“Z”) is present and the crosslinkable nucleotide is incorporated, and FIG. 7B illustrates an example where the SNP is absent and no crosslinkable nucleotide is incorporated. The alternative sequence of the region of interest in FIG. 7B comprises a different nucleobase at the SNP position (depicted as Y). For example, Z could be C, and Y could be any other nucleotide. The region of interest only comprises a single nucleotide having the nucleobase of Z. As shown in FIGS. 7A-7B, the method can comprise contacting the biological sample with a circularizable probe comprising a 3′ arm (1) that hybridizes to a first target sequence in a target nucleic acid in the biological sample, and a 5′ arm (2) that hybridizes to a second target sequence in the target nucleic acid, wherein the first and second target sequence are 3′ and 5′, respectively, to the region of interest. Gap-fill extension can be performed using a nucleotide mixture wherein only the nucleotide complementary to Z (Z′) is functionalized with a crosslinkable moiety, and the other nucleotides are not functionalized with a crosslinkable moiety. When the SNP of interest (Z) is present, the nucleotide functionalized with the crosslinkable nucleotide is incorporated into the circularizable probe, as shown in FIG. 7A. When the SNP of interest is absent and the alternative SNP (Y) is present, the nucleotide functionalized with the crosslinkable moiety is not incorporated in the gap-fill extension. Thus, the circularizable or circularized probe is functionalized with a crosslinkable moiety and is crosslinked only when the SNP of interest is present in the region of interest.

In some embodiments, the method comprises crosslinking the probe or probe set after performing the extension reaction and before performing the ligation. In some embodiments, the method comprises crosslinking the probe or probe set after performing the ligation. In some embodiments, the method comprises: hybridizing a probe comprising hybridization regions that hybridize to regions flanking the region of interest, washing the biological sample to remove non-specifically hybridized probes, extending the 3′ end of the first hybridization region, crosslinking, performing a stringent wash to remove the non-crosslinked probes, ligating the probe, and then detecting the crosslinked ligated probe.

In some embodiments, the method comprises interrogating a plurality of SNPs comprising the same nucleobase of interest using a single extension step. For example, probes can be designed to target regions flanking a plurality of different regions of interest, where the first sequence of the region of interest comprises the particular nucleobase, and alternative sequences of the region of interest do not comprise the particular nucleobase. To interrogate SNPs comprising other nucleobases, the method can comprise re-hybridizing additional probes or probe sets after having detected the first set of probes or probe sets for the first particular nucleobase of interest. Thus, four cycles of probe or probe set hybridization, gap-fill extension, and ligation with different crosslinkable nucleotides used in each of the four cycles could be used to detect SNPS of all four different nucleobases.

### III. Crosslinkable Moieties and Interstrand Crosslinking

In some embodiments, an immobilization oligonucleotide provided herein comprises a crosslinkable moiety for interstrand crosslinking between the immobilization oligonucleotide and the target nucleic acid. In some embodiments, the crosslinkable moiety forms a stable duplex with the target nucleic acid by intercalation. In some embodiments, crosslinkable moiety forms a stable non-covalent crosslink with the target nucleic acid by intercalation. In some cases, the crosslinkable moiety comprises an acridine. In some cases, the crosslinkable moiety comprises a 9-amino-6-chloro-2-methoxyacridine.

In some embodiments, and immobilization oligonucleotide provided herein comprises a crosslinkable moiety for interstrand crosslinking between the immobilization oligonucleotide and the target nucleic acid. In some embodiments, the crosslinkable moiety is or is in a photoreactive nucleotide residue. The hybridization region of the immobilization oligonucleotide may comprise one or more crosslinkable moieties (e.g., photoreactive nucleotide residues). In some embodiments, crosslinking is performed to form an interstrand crosslink between the immobilization oligonucleotide and the target nucleic acid. In some embodiments, the crosslinking occurs in the hybridization region of the immobilization oligonucleotide. In some embodiments, the immobilization oligonucleotide is crosslinked to the target nucleic acid upon activation by providing a stimulus. In some embodiments, the immobilization oligonucleotide is crosslinked to the target nucleic acid via the one or more crosslinkable moieties in the hybridization region. In some aspects, the methods provided herein comprise crosslinking a primary probe or probe set or a product thereof (e.g., an RCP) to a biological sample or matrix. The crosslinkable moiety or moieties may become photo-activated as described in below, in order to crosslink the immobilization oligonucleotide to the target nucleic acid in the biological sample.

In some embodiments, activation of the crosslinkable moiety is light driven and can be performed in aqueous solution. In some embodiments, crosslinking strands of nucleic acid molecules comprise at least one photo-reactive nucleobase. In some embodiments, the crosslinkable moiety is a photo-reactive nucleobase. In some embodiments, the photo-reactive nucleobase can be any modified nucleobase that is capable of forming a crosslink with another nucleobase in an opposite hybridized strand in the presence of light. In some embodiments, the photo-reactive nucleobase can be a modified pyrimidine or purine nucleobase. In some embodiments, the photo reactive nucleobase can comprise a vinyl, acrylate, N-hydroxysuccinimide, amine, carboxylate or thiol chemical group. In some embodiments, the photo-reactive nucleobase comprises a bromo-deoxyuridine. Example photoreactive crosslinkable moieties and photoreactive nucleotides are described, for example, in Elskens and Madder RSC Chem. Biol., 2021, 2, 410-422, the content of which is herein incorporated by reference in its entirety.

In some embodiments, the crosslinkable moiety comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative.

In some embodiments, the crosslinkable moiety comprises a cyanovinylcarbazole moiety. In some embodiments, the crosslinkable moiety comprises a 3-cyanovinylcarbazole (^{CNV}K) nucleoside or 3-cyanovinylcarbazole modified D-threoninol (^{CNV}D). In some embodiments, the crosslinkable moiety comprises 3-cyanovinylcarbazole phosphoramidite. In some embodiments, the crosslinkable moiety comprises a pyranocarbazole. In some embodiments, the crosslinkable moiety comprises a pyranocarbazole (^{PC}X) modified nucleoside or a pyranocarbazole with a D-threoninol instead of a 2′-deoxyribose backbone (^{PCX}D). In some embodiments, the crosslinkable moiety comprises a psoralen or a coumarin. In some embodiments, the nucleotide residues comprising crosslinkable moieties have been attached (e.g., by extension with a polymerase or ligation) to an immobilization oligonucleotide probe that is hybridized to a target nucleic acid within a sample. In some embodiments, the photoreactive nucleotides have been attached to the immobilization oligonucleotide via a linker (e.g., a disulfide linker). In some embodiments, the crosslinkable moiety is a photoreactive nucleotide comprising a universal base.

In some embodiments, the crosslinkable moiety is a pyranocarbazole (^{PC}X) modified nucleoside. The ^{PC}X crosslinking base displays high crosslinking efficiency with a thymine (T) base or a cytosine (C) base that is positioned adjacent to the base on the complementary strand and can be directly incorporated into the DNA hybridization domain itself as a base substitution. In some embodiments, a crosslinking reaction is performed using 400 nm wavelength of light and can be completed within about 10 seconds. In some embodiments, a crosslinking reaction can be completed within 0.1, 0.25, 0.5, 1, 5, or 10 seconds. In some embodiments, a crosslinking reaction can be completed within 0.5, 1, 5, 10, 20, 30, 40, 50, or 60 minutes. In some embodiments, a crosslinking reaction can be completed within 0.5, 1, 2, 3, 4, or 5 minutes. In some embodiments, a crosslinking reaction has negligible effects on bases that neighbor the photoreactive nucleobase. In some embodiments, other photochemical nucleic acid crosslinking agents, including psoralen can be used in combination with the photoreactive nucleobases disclosed herein. In some embodiments, a photo-induced crosslink can be reversed. In some embodiments, a ^{PC}X crosslink can be reversed when exposed to 312305 nm UV light. In some embodiments, the crosslinkable moiety is a photoreactive nucleotide comprising a universal base.

In some embodiments, other photochemical nucleic acid crosslinking agents, including psoralen and psoralen derivatives (e.g., psoralen modified nucleosides) can be used as crosslinkable moieties. Psoralen and psoralen derivatives can be light-activated with a UV-A of 365 nm. Psoralens react with nearby pyrimidine residues. A variety of nucleosides modified with psoralen or psoralen derivatives may be used. For example, click chemistry using a psoralen azide and a nucleosidic alkyne derivative can be used to generate a variety of photoreactive nucleotides. The psoralen can be connected to the nucleotide via a linker, such as a phosphoramidite. Examples of psoralen derivatives comprising phosphoramidite include but are not limited to 6-[4′-(Hydroxymethyl)-4,5′,8-trimethylpsoralen]-hexyl-1-O-(2-cyanoethyl)-(N,N-diisopropyl)-phosphoramidite and 2-[4′-(hydroxymethyl)-4,5′,8-trimethylpsoralen]-ethyl-1-O-(2-cyanoethyl)-(N,N-diisopropyl)-phosphoramidite. In some embodiments, the psoralen or psoralen derivative is conjugated to position 5 of a uridine or pseudouridine (optionally via a linker). In some cases, the psoralen or psoralen derivative is conjugated to the 2′ position of a sugar ring of a uridine or pseudouridine (optionally via a linker). In some embodiments, the psoralen derivative can be an amine-reactive derivative, which can be conjugated to an amine-modified nucleotide (e.g., an aminoallyl uridine or pseudouridine nucleotide).

In some embodiments, a psoralen or psoralen derivative can be crosslinked to a polyA sequence, such as the polyA tail of an mRNA. In some embodiments, the immobilization oligonucleotide comprises an oligodT sequence and a crosslinkable moiety, wherein the crosslinkable moiety is a psoralen or psoralen derivative.

In some embodiments, a psoralen-crosslink (e.g., an interstrand crosslink between the immobilization oligonucleotide and the target nucleic acid) can be reversed when exposed to 254 nm light. In some embodiments, the crosslinkable moiety comprises a C2′ psoralen modification. The crosslinkable moiety can comprise a 5′ psoralen derivative, and can be at the 5′ end of the hybridization region or the 5′ end of the immobilization oligonucleotide. The structure of two example psoralen-modified oligonucleotides (one 5′ modified nucleoside on the left, and one C2′ modified nucleoside on the right) are shown below:

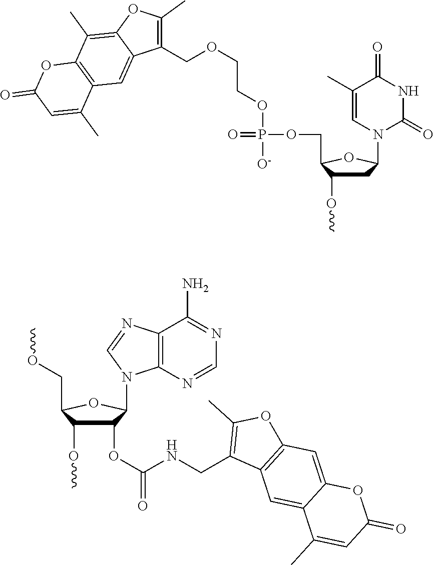

In some embodiments, the crosslinkable moiety is or is linked to a photoactivatable nucleotide, wherein the photoactivatable nucleotide is a universal base such as a pseudouridine modified with a photoreactive moiety (e.g. a psoralen).

In some embodiments, when the immobilization oligonucleotide comprises ^{CNV}K, rapid photo cross-linking to pyrimidines in the complementary strand (DNA or RNA) can be induced at one wavelength and rapid reversal of the cross-link is possible at a second wavelength if desired. Neither wavelength has the potential to cause significant DNA damage and neither interfere with the wavelengths used to excite the fluorophores used during subsequent analysis, such as decoding barcode sequences in situ. Once cross-linked, the UV melting temperature of the duplex may be raised by around 30° C./^{CNV}K moiety relative to the duplex before irradiation and inter-strand crosslinking. The structure of a 3-cyanovinylcarbazole phosphoramidite is shown below:

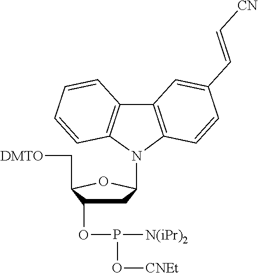

### 5′-O-(4,4′-Dimethoxytrityl)-1′-(3-cyanovinylcarbazol-9-yl)-2′-deoxy-β-D-ribofuranosyl-3′-[(2-cyanoethyl)-(N,N-diisopropyl)]-phosphoramidite

The ^{CNV}K crosslinking base displays high crosslinking efficiency with a thymine (T) base that is positioned adjacent to the base on the opposite hybridized strand in the target nucleic acid (e.g., the complementary strand) (Ultrafast reversible photo-cross-linking reaction: toward in situ DNA manipulation. Org. Lett. 10, 3227-3230 (2008)) and can be directly incorporated into the DNA hybridization domain itself as a base substitution, as shown below in light-directed reaction between a ^{CNV}K base modification and a thymine base to produce a crosslinked nucleic acid.

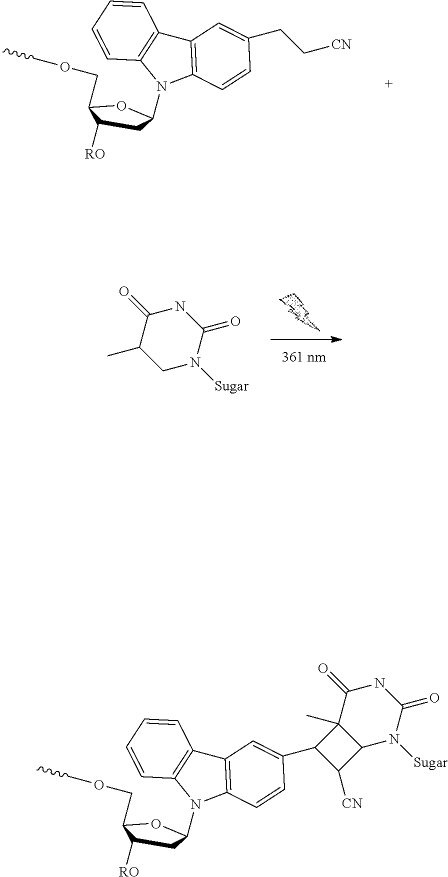

In some embodiments, a crosslinking reaction is performed using 365 nm wavelength of light and can be completed within about 1 second. In some embodiments, a crosslinking reaction can be performed using any wavelength of visible or ultraviolet light. In some embodiments, a crosslinking reaction can be completed within 0.1, 0.25, 0.5, 1, 5, or 10 seconds. In some embodiments, a crosslinking reaction can be completed within 20, 30, 40, 50, or 60 seconds. In some embodiments, the method comprises irradiating the biological sample with UV light, such as a 350-400 nm wavelength of light, for between 10 seconds and 10 minutes, between 10 seconds and 5 minutes, between 10 seconds and 2 minutes, between 10 seconds and 1 minute, between 30 seconds and 1 minute, or between 30 seconds and 5 minutes. In some embodiments, a crosslinking reaction can be completed within 0.5, 1, 5, 10, 20, 30, 40, 50, or 60 minutes. In some embodiments, a crosslinking reaction has negligible effects on bases that neighbor the photoreactive nucleobase. In some embodiments, other photochemical nucleic acid crosslinking agents, including psoralen and coumarin can be used in combination with the photoreactive nucleobases disclosed herein.

In some embodiments, the crosslinkable moiety comprises a coumarin and the photoactivation comprises irradiating the biological sample using a 350 nm wavelength of light. In some embodiments, the crosslinkable moiety comprises a psoralen and the photoactivation comprises irradiating the biological sample using a 365 nm wavelength of light. In some embodiments, the crosslinkable moiety comprises a ^{CNV}K or ^{CNV}D and the photoactivation comprises irradiating the biological sample using a 365 nm wavelength of light. In some embodiments, the crosslinkable moiety comprises a ^{PC}X or ^{PCX}D and the photoactivation comprises irradiating the biological sample using a 400 nm wavelength of light. In some embodiments, the crosslinkable moiety comprises a diazirine and the photoactivation comprises irradiating the biological sample using a 365 nm wavelength of light. In some embodiments, the crosslinkable moiety comprises a thiouridine and the photoactivation comprises irradiating the biological sample using a 365 nm wavelength of light.

In some embodiments, a photo-induced crosslink can be reversed. In some embodiments, a vinylcarbazole (e.g., ^{CNV}K ^{CNV}D, ^{PC}X, or ^{PCX}D) crosslink can be reversed when exposed to 305 nm UV light. In some embodiments, a vinylcarbazole (e.g., ^{CNV}K, ^{CNV}D, ^{PC}X, or ^{PCX}D) crosslink can be reversed when exposed to 312 nm light. In some embodiments, a psoralen crosslink can be reversed when exposed to 254 nm light. In some embodiments, a coumarin crosslink can be reversed when exposed to 254 nm light.

In some embodiments, the crosslinkable moiety is a photoactivatable nucleotide comprising a coumarin and hybridizes to a thymine (T) base in the complementary strand. In some embodiments, the photoactivatable nucleotide comprises a psoralen and hybridizes to a C, T, or U base in the complementary strand. In some embodiments, the photoactivatable nucleotide comprises a vinylcarbanazole and hybridizes to a C, T, or U base in the complementary strand. In some embodiments, the photoactivatable nucleotide comprises a universal or random base.

In some embodiments, the crosslinkable moiety crosslinks to an adenine (A) nucleobase in the strand of the target nucleic acid hybridized to the immobilization oligonucleotide. In some embodiments, the immobilization oligonucleotide comprises a plurality of thymidine and/or uridine residues, optionally wherein one or more of the residues are modified with a psoralen. In some embodiments, the crosslinkable moiety comprises a psoralen capable of crosslinking to an adenine in the hybridized nucleic acid strand. In some embodiments, the crosslinkable moiety comprises a methacrylate C6 phosphoramidite, a psoralen C2 phosphoramidite, a 5-I-dU-CE phosphoramidite, or a 4-Thio-dT-CE phosphoramidite. In some embodiments, the crosslinkable moiety comprises a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In some embodiments, crosslinkable moiety comprises a 5′-Dimethoxytrityl-5-iodo-2′-deoxyUridine,3′-[(2-cyanoethyl)-(N,N-diisopropyl)]-phosphoramidite (5-I-dU-CE phosphoramidite).

The structure of a psoralen C2 phosphoramidite crosslinkable moiety is shown below:

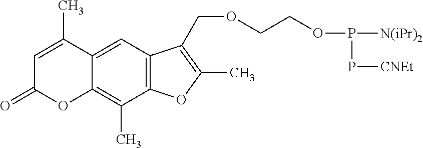

The structure of an example 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite) crosslinkable moiety is shown below:

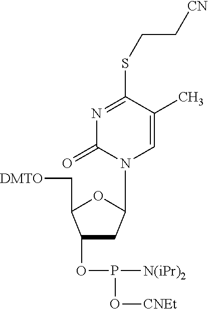

The structure of an example 5′-Dimethoxytrityl-5-iodo-2′-deoxyUridine,3′-[(2-cyanoethyl)-(N,N-diisopropyl)]-phosphoramidite (5-I-dU-CE phosphoramidite) crosslinkable moiety is shown below:

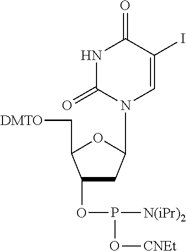

In some embodiments, the target nucleic acid hybridized to the immobilization oligonucleotide is immobilized within the biological sample or matrix generally at the location of the target nucleic acid hybridized by the immobilization oligonucleotide, thereby creating a localized nucleic acid concatemer comprising the target nucleic acid. In some cases, the target nucleic acid is covalently linked to the immobilization oligonucleotide by interstrand crosslinking using the crosslinkable moiety of the immobilization oligonucleotide. In some embodiments, the target nucleic acid is immobilized within the biological sample or matrix by covalent or noncovalent bonding between the attachment moiety and a molecule in the biological sample or the matrix. In some embodiments, by being immobilized to the biological sample or matrix, the size and spatial relationship of the original target nucleic acids is maintained. In some embodiments, a primary probe or amplification product thereof is also immobilized in the biological sample or matrix, such as using a primer comprising functional moiety for attachment to the biological sample or matrix (e.g., for generating a rolling circle amplification product) and/or by incorporating one or more crosslinkable moieties into a rolling circle amplification product. In some embodiments, by being immobilized to the target nucleic acid, such as by covalent bonding or cross-linking, the immobilization oligonucleotide is resistant to movement or dehybridization under mechanical stress. In some embodiments, by being immobilized in the biological sample or matrix, such as by covalent bonding or cross-linking, the rolling circle amplification product is also resistant to movement or unraveling under mechanical stress.

The photoreactive nucleotides may be photo-activated by UV light, such as a 350-400 nm wavelength of light, to photo-activate and crosslink the crosslinkable moiety of the hybridized immobilization oligonucleotide to the target nucleic acid. In some embodiments, the crosslinkable moiety is crosslinked to the complementary strand at a 355 nm wavelength of light. In some embodiments, the purine bases of the target nucleic acid are unreactive to photo-activated crosslinking. In some embodiments, the pyrimidine bases of the complementary strand are reactive to photo-activated crosslinking. In some embodiments, the purine bases of the target nucleic acid are reactive to crosslinking (e.g., to a psoralen, 5-I-dU-CE, 4-Thio-dT-CE, or any other crosslinkable moiety configured to crosslink with nucleobases including adenine).

The photo-activated crosslinking step may be optimized to prevent DNA damage. In some embodiments, the photo-activated crosslinking does not cause significant DNA damage. In some embodiments, the photo-activated crosslinking of the nucleic acid concatemer or the oligonucleotide hybridization region to the complementary strand increases the UV melting temperature of the duplex compared to prior to the crosslinking. In some embodiments, the UV melting temperature is increased by about 30° C. per photoreactive nucleotide in the hybridization region. This increase in melting temperature allows the nucleic acid concatemer to be immobilized to the complementary strand, thereby maintaining spatial fidelity during downstream analyses.

In some embodiments, the photo-activated crosslinking is reversible. In some embodiments, the photo-activated crosslinking is partially reversible. In some embodiments, the photo-activated crosslinking is completely reversible. In some embodiments, the reverse crosslinking comprises exposing the sample to UV light, such as between about 310 nm and 315 nm wavelength of light. In some embodiments, the reverse crosslinking comprises exposing the sample to 312 nm wavelength of light. In some embodiments, the reverse crosslinking comprises about 3 minutes.

The photo-activated crosslinking and/or immobilization of the target nucleic acid maintains spatial orientation of the target nucleic acid relative to the biological sample or matrix in the presence of denaturing agents. In some embodiments, the method further comprises processing the biological sample comprising the immobilized target nucleic acid. In some embodiments, the processing comprises subjecting the biological sample comprising the crosslinked nucleic acid concatemer to a denaturing condition. In some embodiments, the denaturing condition comprises a contacting the biological sample with a denaturing agent and/or heating the biological sample. In some embodiments, the denaturing agent comprises formamide, guanidine, sodium salicylate, dimethyl sulfoxide (DMSO), ethylene carbonate, propylene glycol, or urea. In some embodiments, the denaturing agent comprises formamide. In some embodiments, the denaturing comprises heating the biological sample to disrupt base pairing between the nucleic acid concatemer and the complementary strand. In some embodiments, the biological sample is denatured upon heating above about 80° C. In some embodiments, the biological sample is subjected to repeated cycles of washes that may include denaturing conditions.

### IV. Attachment to the Biological Sample or Matrix

In some embodiments, an immobilization oligonucleotide according to the present disclosure comprises an attachment moiety that can be attached to the biological sample (e.g., to another molecule in the biological sample) or to a matrix. In some embodiments, the attachment moiety is attached to a protein in the biological sample (e.g., by crosslinking). In some embodiments, the protein is in close proximity to a target nucleic acid. In some embodiments, the attachment moiety is attached to an anchoring moiety in a matrix embedding the biological sample. The attachment can be covalent (e.g., crosslinking) or non-covalent (e.g., interaction between a ligand-ligand binding pair).

In some embodiments, the attachment moiety is a reactive group. Example reactive groups for attachment to a biological sample or matrix include, but are not limited to, an amine, a thiol, an azide, an alkyne, a nitrone, an alkene, a tetrazine, a tetrazole, an acrydite or other click reactive group. In some embodiments, the attachment moiety is an acrydite moiety (e.g., as shown in FIG. 1C). In some embodiments, the acrydite is a C6 methacrylate. In some embodiments, the attachment moiety is a methacrylate C6 phosphoramidite.

In some embodiments, the attachment moiety can react with a cross-linker. In some embodiments, the attachment moiety can be part of a ligand-ligand binding pair. Examples of attachment moieties include an amine, amine reactive groups, acrydite, an acrydite modified entity, alkyne, biotin, azide, thiol, and a thiol-modified entity and entities suitable for click chemistry techniques. Biotin, or a derivative thereof, may be used as a matrix attachment moiety when the matrix includes an avidin/streptavidin derivative or an anti-biotin antibody (e.g., a detectably labelled antibody). Similarly, biotin, or a derivative thereof, may be used as a biological sample attachment moiety when the biological sample includes an avidin/streptavidin derivative or an anti-biotin antibody (e.g., a detectably labelled antibody). In some embodiments, a polyacrylamide gel matrix is co-polymerized with acrydite-modified streptavidin. In some instances, biotin or an avidin/streptavidin derivative is attached to the matrix after the matrix is formed. In one example, a polyacrylamide gel matrix is co-polymerized with acrydite-modified streptavidin monomers and biotinylated immobilization oligonucleotides, using a suitable acrylamide:bis-acrylamide ratio to control the cross-linking density. Digoxigenin may be used as a matrix attachment moiety and subsequently bound by an anti-digoxigenin antibody attached to the matrix. An aminoallyl-dUTP residue may be an attachment moiety incorporated into an immobilization oligonucleotide and subsequently coupled to an N-hydroxy succinimide which may be incorporated into the matrix. In some embodiments, a Dibenzocyclooctyne (DBCO)-azide attachment moiety can be used for matrix attachment. In some embodiments, a DBCO attachment moiety is incorporated into the immobilization oligonucleotide, and the matrix comprises an azide. In some embodiments, the DBCO is reacted with the azide in a strain promoted alkyne-azide cycloaddition (SPAAC). In some embodiments, an analyte is attached to the matrix using acrydite (e.g., by copolymerization), NHS ester (e.g., coupling of an NHS ester linked to the analyte with an amine on the matrix), DBCO (e.g., coupling a DBCO in the analyte to an azide on the matrix), sulfhydryl (e.g, coupling a sulfhydryl in or associate with the analyte to a maleimide on matrix), amine (e.g., coupling an amine in or associated with the analyte to a carboxyl on the matrix, or coupling a carboxyl in or associated with the analyte to an amine on the matrix). In general, any member of a conjugate pair or reactive pair may be used as an attachment moiety to attach an immobilization oligonucleotide to a matrix.

Attachment moieties for attachment to a matrix include chemical cross-linking agents. Cross-linking agents typically contain at least two reactive groups that are reactive towards numerous groups, including, but not limited to, sulfhydryls and amines, and create chemical covalent bonds between two or more molecules. Attachment moieties include cross-linking agents such as primary amines, carboxyls, sulfhydryls, carbohydrates and carboxylic acids. Protein molecules have many of these functional groups and therefore proteins and peptides can be readily conjugated using cross-linking agents. Cross-linking agents are commercially available (Thermo Scientific (Rockford, IL)). In the case of crosslinking, the matrix attachment moiety may attached to modified dNTP or dUTP in the immobilization oligonucleotide, or to both. Suitable example cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. Such spacer moieties may be functionalized. Such spacer moieties may be chemically stable. Suitable example spacer moieties include polyethylene glycol, carbon spacers, cleavable (e.g., photo-cleavable or chemically cleavable) spacers and other spacers and the like.

In some embodiments, the methods provided herein comprise attaching a primary probe or probe set or a product thereof (e.g., an RCP generated from a circular or circularized probe or probe set) to the biological sample or matrix. In some embodiments, an rolling circle amplification of the circular or circularized probe set (e.g., a primary probe or probe set) can be performed using a primer comprising a functional moiety for attachment to the biological sample or matrix. The functional moiety can be any of the attachment moieties described above. In some cases, the functional moiety of the primer is orthogonal to the attachment moiety of the immobilization oligonucleotide. In some embodiments, the method comprises contacting the biological sample or matrix with a nucleotide mixture comprising one or more modified crosslinkable nucleotides for incorporation into the RCP. In some embodiments, the modified crosslinkable nucleotides can be functionally orthogonal to the attachment moiety of the immobilization oligonucleotide. In some cases, the matrix is a multifunctional matrix (e.g., a multifunctional hydrogel), comprising a first anchoring moiety for attachment to the attachment moiety of the immobilization oligonucleotide, and a second anchoring moiety for attachment to the functional moiety of the RCA primer and/or the crosslinkable nucleotides incorporated into the RCP. For example, the first anchoring moiety can be an acrydite moiety for covalent attachment of a methacrylate attachment moiety of the immobilization oligonucleotide, and the second anchoring moiety can be a methylsulfone moiety for attachment to a thiolated functional moiety in the RCA primer and/or alpha-thiol nucleotides incorporated into the RCP. In some cases, the attachment moiety in the immobilization oligonucleotide and the functional moiety in the primer and/or crosslinkable nucleotides can be the same.

In some embodiments, an RCP is generated in the biological sample opr matrix, wherein the RCP comprises modified nucleotide residues having functional linkage groups for tethering to a matrix, such as acrylamide or click-reactive groups, enabling the products of amplification to be spatially immobilized via covalent gel linkages. According to one aspect, the functional linkages can be incorporated during amplification using nucleotide analogs, including amino-allyl dUTP, 5-TCOPEG4-dUTP, C8-Alkyne-dUTP, 5-Azidomethyl-dUTP, 5-Vinyl-dUTP, 5-Ethynyl dUTP, or a combination thereof. According to a separate aspect, for amplification methods using one or more primers, one or more of the primers can comprise a functional linkage group for tethering to a matrix, e.g., solid-state.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

Matrix forming materials include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions.

According to one aspect, a matrix-forming material can be introduced into a cell. The cells are fixed with formaldehyde and then immersed in ethanol to disrupt the lipid membrane. The matrix forming reagents are added to the sample and are allowed to permeate throughout the cell. A polymerization inducing catalyst, UV or functional cross-linkers are then added to allow the formation of a gel matrix. The un-incorporated material is washed out and any remaining functionally reactive group is quenched. Example cells include any cell, human or otherwise, including diseased cells or healthy cells. Certain cells include human cells, non-human cells, human stem cells, mouse stem cells, primary cell lines, immortalized cell lines, primary and immortalized fibroblasts, HeLa cells and neurons.

According to one aspect, a matrix-forming material can be used to encapsulate a biological sample, such as a tissue sample. The formalin-fixed embedded tissues on glass slides are incubated with xylene and washed using ethanol to remove the embedding wax. They are then treated with Proteinase K to permeabilized the tissue. A polymerization inducing catalyst, UV or functional cross-linkers are then added to allow the formation of a gel matrix. The un-incorporated material is washed out and any remaining functionally reactive group is quenched.

According to one aspect, the matrix-forming material forms a three dimensional matrix including a plurality of analytes and/or target nucleic acid molecules while maintaining the spatial relationship of the analytes and/or target nucleic acid molecules. In some embodiments, the immobilization oligonucleotide hybridized to the target nucleic acid is used to attach the target nucleic acid covalently or non-covalently to the matrix. In some embodiments, the immobilization oligonucleotide hybridizes non-specifically to a plurality of target nucleic acids. In some embodiments, the immobilization oligonucleotide hybridizes to a complementary sequence present in a plurality of target nucleic acid molecules (e.g., to a polyA sequence common among a plurality of target mRNA molecules). In this aspect, the plurality of target nucleic acids are immobilized within the matrix material. The plurality of target nucleic acid molecules may be immobilized within the matrix material by co-polymerization of the immobilization oligonucleotide with the matrix-forming material. The immobilization oligonucleotides may also be immobilized within the matrix by covalent attachment or through ligand-ligand interaction to the matrix.

According to one aspect, the matrix is sufficiently optically transparent or otherwise has optical properties suitable for deep three dimensional imaging for high throughput information readout, such as for detection using labeled probes (e.g., fluorescently labeled probes).

According to one aspect, the matrix is porous thereby allowing the introduction of reagents (e.g., primary probes or probe sets, intermediate probes, and/or detectably labeled probes) into the matrix at the site of a target nucleic acid molecule immobilized in the matrix. Additional control over the molecular sieve size and density is achieved by adding additional cross-linkers such as functionalized polyethylene glycols. According to one aspect, the target nucleic acid molecules are readily accessed by probes, enzymes, and other reagents with rapid kinetics. Porosity can result from polymerization and/or crosslinking of molecules used to make the matrix material. The diffusion property within the gel matrix is largely a function of the pore size. The molecular sieve size is chosen to allow for rapid diffusion of enzymes, oligonucleotides, formamide and other buffers used for amplification and detection (>50-nm). The molecular sieve size is also chosen so that large DNA or RNA amplicons do not readily diffuse within the matrix (<500-nm). The porosity is controlled by changing the cross-linking density, the chain lengths and the percentage of co-polymerized branching monomers

In some embodiments, the biological sample is immobilized in the hydrogel via cross-linking of the polymer material that forms the hydrogel. Cross-linking can be performed chemically and/or photochemically, or alternatively by any other hydrogel-formation.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

A matrix can be formed using a photopolymerization. Photopolymerization can use photons to initiate a polymerization reaction. The photopolymerization reaction can be initiated by a single-photon or a multiphoton excitation system as described elsewhere herein. Light can be manipulated such to form specific 2D or 3D patterns and be used to initiate the photopolymerization reaction. This can be used to construct a particular shape or pattern for the 3D matrix such that the matrix is generated in one part of the cell or cell derivative but not generated in another part of the cell or cell derivative. Light and patterns of light can be generated by spatial light modulators, such as a digital spatial light modulator. The spatial light modulators can employ a transmissive liquid crystal, reflective liquid crystal on silicon (LCOS), digital light processing, a digital micromirror device (DMD), or a combination thereof.

The fixative/hydrogel composition can comprise any hydrogel subunits, such as, but not limited to, poly(ethylene glycol) and derivatives thereof (e.g., PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose and the like. Agents such as hydrophilic nanoparticles, e.g., poly-lactic acid (PLA), poly-glycolic acid (PLG), poly (lactic-co-glycolic acid) (PLGA), polystyrene, poly(dimethylsiloxane) (PDMS), etc. can be used to improve the permeability of the hydrogel while maintaining patternability. Materials such as block copolymers of PEG, degradable PEO, poly (lactic acid) (PLA), and other similar materials can be used to add specific properties to the hydrogel. Crosslinkers (e.g., bis-acrylamide, diazirine, etc.) and initiators (e.g., azobisisobutyronitrile (AIBN), riboflavin, L-arginine, etc.) can be included to promote covalent bonding between interacting macromolecules in later polymerization.

Examples of suitable attachment moieties or functional moieties include electrophiles or nucleophiles that can form a covalent linkage by reaction with a corresponding nucleophile or electrophile, respectively, on the substrate of interest. Non-limiting examples of suitable electrophilic reactive groups can include, for example, esters including activated esters (such as, for example, succinimidyl esters), amides, acrylamides, acridines, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups can include, for example, amines, anilines, thiols, alcohols, phenols, hyrazines, hydroxylamines, carboxylic acids, glycols, heterocycles, and the like. Further non-limiting examples of functional moieties include acrydite, biotin, alkyne, and amine groups. In some embodiments, the immobilization oligonucleotide or other oligonucleotides (such as a primer for RCA) comprise an attachment/functional moiety. Functional moieties can be incorporated into oligonucleotides by, for example, incorporation during chemical oligonucleotide synthesis, or chemical conjugation to an oligonucleotide.

In some embodiments, after attachment of the immobilization oligonucleotide to the matrix, the matrix can be partially or substantially cleared of certain species or classes of biomolecules, such as lipids and proteins, as by use of detergent and/or protease reagents. According to some aspects of the present disclosure, the sample can be cleared using a detergent solution, such as Triton-X or SDS. The detergent can interact with the molecules allowing the molecules to be washed out or removed. Other non-limiting examples of detergents include Triton X-100, Triton X-114, Tween-20, Tween 80, saponin, CHAPS, and NP-40. According to some aspects of the present disclosure, the sample can be cleared using a protease reaction, such as Proteinase K. The protease can cleave or digest proteins such that the fragments or amino acids can be removed. According to some aspects of the present disclosure, the extracellular matrix can be substantially cleared using one or more specific or nonspecific proteases. Other non-limiting examples of protease include trypsin, chemotrypsin, papain, thrombin, and pepsin.

In some embodiments, the biological sample or the matrix is immobilized onto a solid substrate, such as glass or plastic, facilitating handling and reagent exchange. According to one aspect, a matrix can be affixed to a glass slide via oxysilane-functionalization with acrylamide- or free-radical-polymerizing groups, such as methacryloxypropyltrimethoxysilane. The 3D matrix can be free-floating or otherwise not attached to a solid substrate.

Additional methods and aspects of hydrogel embedding of biological samples are described for example in Chen et al., Science 347(6221):543-548, 2015, the content of which is herein incorporated by reference in its entirety.

In some embodiments, provided herein is a method of processing a biological sample comprising contacting the biological sample with a first species of matrix-forming material, polymerizing the first species of matrix-forming material to form a first matrix, contacting the first matrix with a second species of matrix-forming material, and polymerizing the second species of matrix-forming material to form a second matrix. In some embodiments, the first matrix is covalently attached to the second matrix. In some embodiments, the first matrix is non-covalently attached to the second matrix. In some embodiments, the first matrix is not attached to the second matrix. In some embodiments, the first species of matrix-forming material is different from the second species of matrix-forming material. In some embodiments, the first species of matrix-forming material is the same as the second species of matrix-forming material. In some embodiments, the method comprises delivering one or more reagents to the biological sample embedded in the first matrix before contacting the first matrix with the second species of matrix-forming material. In certain embodiments, the method comprises contacting the biological sample with any of the immobilization oligonucleotides disclosed herein, wherein the immobilization oligonucleotide is attached to the first matrix before contacting the biological sample with the second matrix. In some embodiments, the immobilization oligonucleotide is attached to the first matrix and to the second matrix. In some embodiments, the immobilization oligonucleotide is attached to the first matrix and to the second matrix using orthogonal attachment chemistries.

In some embodiments, the first attachment moiety is attached to a first anchoring moiety in the biological sample or a matrix embedding the biological sample, wherein the first attachment moiety and the first anchoring moiety are a ligand-ligand binding pair, or functional moieties that can react with each other. In some embodiments, the second attachment moiety is attached to a second anchoring moiety in the biological sample or a matrix embedding the biological sample, wherein the second attachment moiety and the second anchoring moiety are a ligand-ligand binding pair, or functional moieties that can react with each other. In some embodiments, the first attachment moiety and the first anchoring moiety react with each other via a first chemistry, and the second attachment moiety and the second anchoring moiety react with each other via a second chemistry, wherein the first and second chemistries are orthogonal. In certain embodiments, the first attachment moiety and the second attachment moiety are attached to the same matrix embedding the biological sample using orthogonal reaction chemistries. In certain embodiments, the first attachment moiety is attached to a first matrix embedding the biological sample and the second attachment moiety is attached to a second matrix embedding the biological sample.

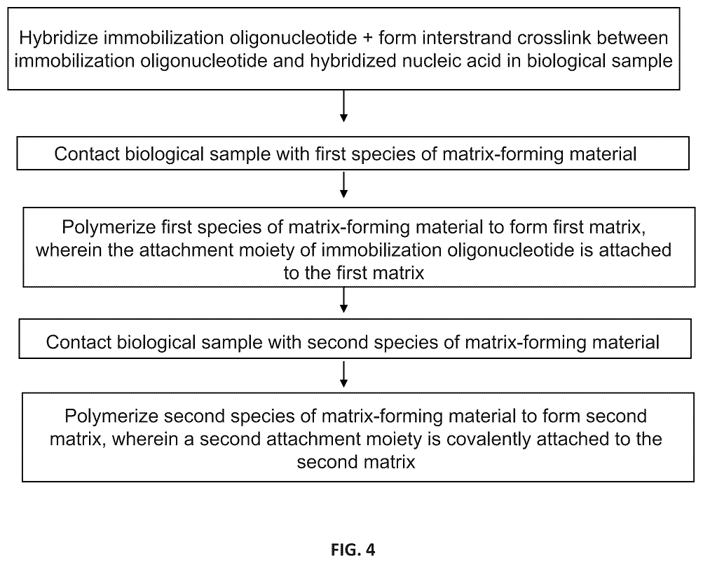

An example of some embodiments of a workflow comprising iterative infusion of matrix forming materials and matrix formation is provided in FIG. 4. In some embodiments, a method of processing a biological sample comprises hybridizing an immobilization oligonucleotide (e.g., any immobilization oligonucleotide described herein) to a nucleic acid in the biological sample. In some cases, the immobilization oligonucleotide comprises a random sequence or a sequence of universal bases that hybridizes to a nucleic acid in the biological sample. In some embodiments, the immobilization oligonucleotide hybridizes to a target sequence present in a plurality of target nucleic acids (e.g., a polyA tail in mRNAs). In some cases, the immobilization oligonucleotide comprises a poly-T sequence (e.g., a sequence of at least 5, 10, 15, or 20 Ts) that hybridizes to a polyA tail present in RNA transcripts in the biological sample. In some embodiments, the immobilization oligonucleotide hybridizes to a target sequence that is unique to a target nucleic acid. In some embodiments, the immobilization oligonucleotide comprises a crosslinkable moiety capable of interstrand crosslinking with a hybridized nucleic acid. In some embodiments, the immobilization oligonucleotide comprises a poly-T sequence, and the crosslinkable moiety comprises a psoralen. In some embodiments, the immobilization oligonucleotide comprises a random sequence or a sequence of universal bases, and the crosslinkable moiety comprises a cyanovinylcarbazole moiety. In some embodiments, the method comprises forming an interstrand crosslink between the immobilization oligonucleotide and the target nucleic acid (e.g., in an embodiment as illustrated in FIG. 4).

In some cases, the method comprises contacting the biological sample with a first species of matrix forming material after hybridizing an immobilization oligonucleotide to a nucleic acid in the biological sample and forming an interstrand crosslink between the immobilization oligonucleotide and the hybridized nucleic acid in the biological sample. In some embodiment, the first species of matrix-forming material comprises a functional moiety for attachment to an attachment moiety of the immobilization oligonucleotide. For example, the attachment moiety can comprise a methacrylate, and the first matrix-forming material can comprise acrylamide monomers. An acrydite moiety of the first matrix forming material can serve as an anchoring moiety for covalent attachment of a methacrylate attachment moiety of the immobilization oligonucleotide. In some embodiments, after polymerizing the first species of matrix-forming material to form the first matrix attached to the first attachment moiety of the immobilization oligonucleotide, the method comprises contacting the biological sample with a second species of matrix-forming material. In some embodiments, the second species of matrix forming material is different from the first species of matrix-forming material. In some embodiments, the second species of matrix forming material is different from the first species of matrix-forming material. For example, in some embodiments, the first species matrix-forming material comprises acrylamide monomers, and the second species of matrix-forming material comprises a methylsulfone moiety. In other embodiments, the first species matrix-forming material comprises a methylsulfone moiety, and the second species of matrix-forming material comprises acrylamide monomers.

In some cases, the method comprises contacting the biological sample with a first matrix forming material after hybridizing an immobilization oligonucleotide to a nucleic acid in the biological sample and forming an interstrand crosslink between the immobilization oligonucleotide and the hybridized nucleic acid in the biological sample. In some embodiment, the first species of matrix-forming material comprises a functional moiety for attachment to an attachment moiety of the immobilization oligonucleotide. For example, the attachment moiety can comprise a methacrylate, and the first matrix-forming material can comprise acrylamide monomers. An acrydite moiety of the first matrix forming material can serve as an anchoring moiety for covalent attachment of a methacrylate attachment moiety of the immobilization oligonucleotide. In some embodiments, after polymerizing the first species of matrix-forming material to form the first matrix attached to the first attachment moiety of the immobilization oligonucleotide, the method comprises contacting the biological sample with a second matrix-forming material. In some embodiments, the first matrix-forming material and the second matrix-forming material are the same type of matrix-forming material. In some embodiments, the first and second matrix are attached to each other (e.g., forming a double network motif). For example, in some embodiments, the first matrix-forming material comprises acrylamide monomers, and the second species of matrix-forming material acrylamide moieties. In some embodiments, the first matrix-forming material and the second matrix-forming material are different, but the two matrices are non-covalently attached to each other.

In some embodiments, the method comprises polymerizing the second species of matrix-forming material to form a second matrix, wherein a second attachment moiety is covalently or non-covalently attached to the second matrix. The second attachment moiety can be part of the immobilization oligonucleotide. In some embodiments, the second attachment moiety is part of a separate nucleic acid molecule, such as a modified primer used to generate an RCA product from a circular or circularized probe hybridized to the target nucleic acid in the biological sample. In some embodiments, the second attachment moiety is attached to a labeling agent, such as an attachment moiety in an oligonucleotide reporter attached to an antibody that binds to a protein analyte in the biological sample. In some embodiments, the second attachment moiety is incorporated into an extension or amplification product generated in the biological sample using modified nucleotides comprising the second attachment moiety, such as alpha-thiol nucleotides.

In some embodiments, polymerization of the first species of matrix-forming material to form the first matrix attached to the first attachment moiety of the immobilization oligonucleotide and polymerization of the second species of matrix-forming material to form a second matrix, wherein a second attachment moiety is covalently attached to the second matrix is performed at the same time. In some embodiments, polymerization of both the first and second species of matrix-forming materials to form the first and second matrix is performed using compatible species of matrix-forming materials.

In some embodiments, provided herein is a method of processing a biological sample comprising contacting the biological sample with an immobilization oligonucleotide comprising: an oligo deoxythymidine (oligo dT) sequence, a crosslinkable moiety comprising a psoralen, and an attachment moiety comprising methacrylate. In some embodiments, a plurality of the immobilization oligonucleotide hybridize to the polyA tail of an mRNA. In certain embodiments, the method then comprises washing the biological sample to remove unbound immobilization oligonucleotides, and exposing the sample to UV light to crosslink the hybridized immobilization oligonucleotides to their hybridized nucleic acid(s). In certain embodiments, acrylamide monomers (a first species of matrix-forming material) are contacted with the biological sample, and allowed to polymerize throughout the tissue and covalently incorporate the methacrylate moiety on the immobilization oligonucleotide. In some cases, a plurality of immobilization oligonucleotides are crosslinked to an individual mRNA and are covalently attached to the matrix, linking the mRNA to the matrix at multiple attachment points. In some embodiments, the method comprises clearing the biological sample after attaching the mRNA to the matrix using the immobilization oligonucleotide. In certain embodiments, clearing comprises contacting the biological sample with protease K and/or a detergent such as sodium dodecyl sulfate (SDS). In some embodiments, clearing the biological sample comprises removing lipids and/or proteins from the biological sample. In some embodiments, nuclear structures (or a portion thereof) remain in the biological sample after clearing. In some embodiments, the spatiality of the mRNA remains intact.

In some embodiments, clearing the biological sample may be performed to remove proteins from the tethered RNA. In some embodiments, biological sample is treated to remove ribosomes from the RNA. For example, the treatment to remove proteins is performed after polymerizing the matrix-forming material to form a matrix and attaching the mRNA to the matrix using the immobilization oligonucleotides described herein. In some embodiments, the method comprises treating the biological sample embedded in the three-dimensional polymerized matrix with a detergent and a protease. In some cases, the detergent comprises SDS and the protease comprises proteinase K. In some embodiments, the method comprises treating the biological sample embedded in the three-dimensional polymerized matrix with 50 to 500 μg/mL proteinase K, 100 to 400 μg/mL proteinase K, 150 to 300 μg/mL proteinase K, or 150 to 250 μg/mL proteinase K. In some embodiments, the method comprises treating the biological sample embedded in the three-dimensional polymerized matrix with about 200 μg/mL proteinase K. In some embodiments, the method comprises treating the biological sample embedded in the three-dimensional polymerized matrix with 0.5% to 2% SDS, 0.5% to 1% SDS, 1% to 2% SDS or about 0.8% to 1.2% SDS. In some embodiments, the method comprises treating the biological sample embedded in the three-dimensional polymerized matrix with 1% SDS. In some embodiments, the method may comprise treating the biological sample embedded in the three-dimensional polymerized matrix with a detergent and a protease in a reaction buffer at about pH 8 to pH 9. In some embodiments, the method may comprise treating the biological sample embedded in the three-dimensional polymerized matrix with a detergent and a protease in a reaction buffer at about pH 8.5.

In some embodiments, the method may comprise treating the biological sample embedded in the three-dimensional polymerized matrix with a detergent and a protease in a reaction buffer at about 45° C. to 60° C., at about 45° C. to 55° C., at about 45° C. to 50° C., at about 48° C. to 55° C., at about 48° C. to 52° C., or at about 50° C. to 52° C. In some embodiments, the method may comprise treating the biological sample embedded in the three-dimensional polymerized matrix with a detergent and a protease in a reaction buffer at about 50° C. for at least 2 minutes, at least 3 minutes, or at least 4 minutes. In some embodiments, the method may comprise treating the biological sample embedded in the three-dimensional polymerized matrix with a detergent and a protease in a reaction buffer at about 50° C. for no more than 5 minutes, no more than 4 minutes, or no more than 3 minutes. In some instances, the biological sample embedded in the three-dimensional polymerized matrix is treated with 1% SDS and 200 μg/ml PK in PBS pH 8.5 at 50° C. for about 3 minutes.

In certain embodiments, after clearing the biological sample, the method comprises contacting the biological sample in the first matrix with a second species of matrix-forming material comprising a methylsulfone moiety. In certain embodiments, the method comprises hybridizing circularizable probes to target RNAs in the biological sample (e.g., after clearing the biological sample), ligating the circularizable probes to generate circularized probes, and hybridizing a thiolated primer to the circularized probes for RCA. The thiolated primer can be attached to the second matrix formed from the second matrix. In some embodiments, RCA is performed using normal dNTPs with alpha-thiol nucleotides spiked in. The thiol nucleotides can be used by the polymerase (e.g., Phi29 polymerase) as a substrate and incorporated into the RCA product. The thiolated RCA product can be connected to the second matrix via the methylsulfone moieties.

In some embodiments, the first matrix has large pores and weak rigidity relative to the second matrix. In some embodiments, the second matrix comprises smaller pores compared to the first matrix. In some embodiments, the first and the second matrix comprise acrylamide and bis-acrylamide, wherein the amounts of acrylamide and bis-acrylamide in each matrix differs. In some such embodiments, pore size of the first matrix and second matrix can be controlled and tuned by adjusting the ratio of acrylamide to bis-acrylamide in the respective matrices. In one embodiment, the first matrix comprises about 4% acrylamide (by weight) and about 0.2% bis acrylamide (by weight) and the second matrix comprises weight percentages of acrylamide and bis-acrylamide that differ from the first matrix. Increasing or decreasing the acrylamide and bis-acrylamide will alter the degree of crosslinking, pore size and rigidity, hence imbuing each matrix with different properties. In some embodiments, one of matrices comprises a cleavable matrix forming material. In some embodiments, the cleavable matrix forming material is a polyacrylamide gel cross-linker such as N,N′-(1,2-Dihydroxyethylene)bis-acrylamide. The use of a cleavable matrix forming material allows for the selective removal of one of the two matrices. For instance, in one embodiment, the 1-2-diol group of N′-(1,2-Dihydroxyethylene) bis-acrylamide can be cleaved with periodate, hence dissolving the acrylamide type hydrogel. In some embodiments, the diol cleavage proceeds through a cyclic intermediate referred to as a periodate ester. In some embodiments, the cleavage is performed at a temperature of about 0° C. In some embodiments, one of the matrices is comprises a reversible or cleavable cross-linker such as N,N′-(1,2-Dihydroxyethylene)bis-acrylamide, a reversible cross-linker for polyacrylamide gels which produces a gel that is soluble when incubated with periodate (e.g., solubilized by incubation with dilute (2%) periodic acid at room temperature for 1-2 hr). In some embodiments, the method comprises selectively removing one of the matrices by incubating the matrices under conditions that render one of the matrices soluble.

In some embodiments comprising multiple infusions of different species of matrix-forming material, each infusion of different monomers can polymerize in different ways that can add new aspects of rigidity and other interesting properties such as control over swelling, opacity, and/or the refractive index of the matrix. In some aspects, infusing different types of hydrogels into a single experimental system provides a multitude of approaches for immobilization of nucleic acids, labeling agents, and/or amplification products such as RCA products.

In some embodiments, an immobilization oligonucleotide provided herein has multi-functional coupling abilities into different components of a first matrix and a second matrix. In some embodiments, attaching an immobilization oligonucleotide to a first matrix and a second matrix using different attachment moieties gives added spatial rigidity to the molecules bound by the immobilization oligonucleotide. For example, in some cases, an immobilization oligonucleotide provided herein comprises a crosslinkable moiety for interstrand crosslinking with a hybridized nucleic acid, a first attachment moiety for attachment to a first matrix, and a second attachment moiety for attachment to a second matrix. Also provided herein are labeling agents comprising multi-functional oligonucleotides. For example, provided herein is an antibody that has a conjugated oligonucleotide with both an exposed thiol group and an acrydite moiety, which will be linkable into a standard polyacrylamide hydrogel as well as one that includes methylsulfone monomers. In some embodiments, other oligos that contain norbornene and acrydite can be further crosslinked into other added monomer components infused to the matrix by UV exposure. Additionally or alternatively, nucleotides used during amplification (e.g., rolling circle amplification of a circular or circularized probe in the biological sample) may contain amino-allyl residues, and the primer used to amplify can be endowed with 5′ diazirine moieties. The combinations by using this stepwise infusion process are extensive and far easier to realize experimentally than a single hydrogel with multiple functionalities.

### V. Detection and Analysis

In some aspects, the provided methods involve analyzing, e.g., detecting or determining, one or more sequences present in the target nucleic acid, a complex associated with the target nucleic acid, and/or in the nucleic acid concatemers comprising at least one photoreactive nucleotide as described herein. In some embodiments, the detecting comprises hybridizing a primary probe or probe set to the target nucleic acid, and detecting the primary probe or probe set or an amplification product thereof. In some embodiments, the detecting comprises a plurality of repeated cycles of hybridizing and removal of probes to the primary probe or probe set (e.g., as described in Section II.B.) hybridized to the target nucleic acid. In some cases, the target nucleic acid is RNA, and the primary probe or probe set hybridizes to the RNA target nucleic acid. In some embodiments, the primary probe or probe set hybridizes to a target sequence in the target nucleic acid. In some embodiments, the target sequence is specific to the target nucleic acid (e.g., the target sequence can identify the target nucleic acid). In some embodiments, the primary probe or probe set hybridizes to a target sequence in the target nucleic acid, and the immobilization oligonucleotide hybridizes to a common sequence (e.g., a polyA sequence) present in a plurality of different target nucleic acids. In some embodiments, the primary probe or probe set hybridizes to a specific target sequence in the target nucleic acid, and the immobilization oligonucleotide hybridizes non-specifically to the target nucleic acid (e.g., via a hybridization region comprising universal and/or random bases).

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, or sequencing by hybridization).

Various example probe configurations and methods for detecting the primary probe or probe set or a product thereof are illustrated in FIGS. 2A-2C. In some embodiments, the detecting can comprise binding an intermediate probe directly or indirectly to the primary probe or probe set (e.g., as shown in FIG. 2A), binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized primary probe or probe set as a template (e.g., as shown in FIG. 2B). In some embodiments, the method comprises detecting a rolling circle amplification product (RCP) generated using a circular or circularized probe or probe that binds to a primary probe or probe set as a template (e.g., as shown in FIG. 2C). In some embodiments, rolling circle amplification is performed using the immobilizable probe as a primer. In some embodiments, rolling circle amplification is performed using a separate primer. In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method can comprise performing one or more wash steps to remove unbound and/or nonspecifically bound intermediate probe molecules from the primary probes or the products of the primary probes.

In some embodiments, the detecting can comprise: detecting signals associated with detectably labeled probes that are hybridized to barcode regions or complements thereof in the primary probe or probe set or a product thereof (e.g., an RCP); and/or detecting signals associated with detectably labeled probes that are hybridized to intermediate probes which are in turn hybridized to the barcode regions or complements thereof. In some embodiments, the detectably labeled probes can be fluorescently labeled.

In some embodiments, the methods comprise sequencing all or a portion of a primary probe or probe set or an RCP, or detecting a sequence of the primary probe or probe set or RCP, such as one or more barcode sequences present in the primary probe or probe set or RCP. In some embodiments, the sequence of the RCP, or barcode thereof, is indicative of a sequence of the target nucleic acid to which the RCP is hybridized. In some embodiments, the analysis and/or sequence determination comprises sequencing all or a portion of the nucleic acid concatemer and/or in situ hybridization to the RCP. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product).

In some aspects, the provided methods comprise imaging a detectably labeled probe bound directly or indirectly to the primary probe or probe set or product thereof and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, example techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Example fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Example haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Example techniques for in situ sequencing or in in situ sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121, the content is herein incorporated by reference in its entirety).

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing can be performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing can be performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, a barcode sequence of the primary probe or probe set, a product of the primary probe or probe set, or an intermediate probe bound directly or indirectly to the primary probe or probe set or a product thereof is targeted by detectably labeled secondary probe oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Example decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natd. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VI. Samples and Analytes

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, a cell block, a cell pellet, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface. In some embodiments, the biological sample is a cell or tissue sample.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Sample Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. In some embodiments, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., one or more probes or probe sets and one or more immobilization oligonucleotides. In some embodiments, one or more post-fixing step is performed after a hybridization complex comprising a probe and a target is formed in a sample.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences. A post-fixing step may be performed using any suitable fixation reagent disclosed herein, for example, 3% (w/v) paraformaldehyde in DEPC-PBS.

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (ii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

(iii) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### (iv) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix (e.g., as described in Sections II-IV). For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, provided herein is a method of embedding a biological sample in several distinct intertwined matrices (e.g., hydrogel matrices). In some embodiments, multiple distinct matrix-forming materials are designed to react with attachment moieties used during different steps of sample preparation and/or analysis. In some embodiments, each unique hydrogel composition is infused into the previous incarnation of the matrix (e.g., hydrogel) after reaction of the previous matrix with a cognate attachment moiety. In some embodiments, an analyte is immobilized in a first matrix formed using a first species of matrix-forming material. The analyte can be immobilized using any of the crosslinking and/or attachment methods described herein. In some embodiments, the first matrix comprising the analyte is then contacted with a second species of matrix-forming material.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### (v) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (vi) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some embodiments, the target sequence is in or is associated with an analyte. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific subcellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g., a probe or probe set as described in Section II.B.). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Example analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of example labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### VII. Compositions, Kits, and Systems

In some embodiments, disclosed herein is a composition that comprises a complex containing a target nucleic acid, and immobilization oligonucleotide comprising an attachment moiety and a crosslinkable moiety capable of forming an interstrand crosslink with the target nucleic acid. In some embodiments, the complex further comprises a primary probe or probe set hybridized to the target nucleic acid. In some cases, the primary probe or probe set is hybridized to a target sequence in the target nucleic acid, and the immobilization oligonucleotide is hybridized non-specifically to the target nucleic acid or is hybridized to a common sequence among a plurality of target nucleic acids in a biological sample (e.g., a polyA sequence in mRNA molecules). In some embodiments, the composition further comprises one or more modified nucleotides, e.g., any of the photoreactive nucleotides for attachment of an RCP to a biological sample or matrix described in Section IV. Also provided herein are systems for performing the methods provided herein. In some aspects, the system comprises a source for providing a stimulus (e.g., light activation) to initiate crosslinking.

Also provided herein are kits, for example, comprising one or more immobilization oligonucleotides, e.g., any described in Section II, and instructions for performing the methods provided herein. In some embodiments, the kits further comprise one or more reagents for performing the methods provided herein (e.g., one or more photoreactive nucleotides, such as any of the photoreactive nucleotides described in Sections III and IV). In some embodiments, the kits further comprise one or more reagents required for one or more steps comprising hybridization, ligation, extension, detection, sequencing, and/or sample preparation as described herein. In some embodiments, any or all of the oligonucleotides are DNA molecules. In some embodiments, the target nucleic acid is a messenger RNA molecule. In some embodiments, the kit comprise at least two different species of matrix-forming materials that can be used to form two different types of matrices. In some cases, the first matrix and second matrix is for interacting with at least two different attachment moieties. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, photoreactive nucleotides, and reagents for additional assays.

### VIII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect single nucleotides of interest in target nucleic acids. In some aspects, the provided embodiments can be used to crosslink the immobilization oligonucleotides via photoreactive nucleotides, e.g., to the hybridized nucleobase in the target nucleic acid, to increase the stability of the target nucleic acid in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### IX. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Example stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### Example

The following example is included for illustrative purposes only and is not intended to limit the scope of the present disclosure.

### Example 1: Immobilization of Target Nucleic Acids Using Bifunctional Immobilization Oligonucleotides

This example provides an example method for analyzing a biological sample wherein a plurality of target nucleic acids are immobilized in the sample using an immobilization oligonucleotide comprising a crosslinkable moiety and an attachment moiety.

In a second aspect, this example provides an example method for detecting the target nucleic acid using a circularizable primary probe or probes set and performing rolling circle amplification (RCA) to generate a rolling circle amplification product (RCP) that is further anchored to a matrix, to further enhance spatial fidelity of the resulting RCP.

A biological sample (e.g., a processed or cleared biological sample, a tissue sample, a sample embedded in a hydrogel, etc.) is contacted with a circularizable probe or probe set (e.g., a padlock probe), and with an immobilization oligonucleotide. An example immobilization oligonucleotide is depicted in FIG. 1C. The immobilization oligonucleotide is bifunctional—it comprises (1) a crosslinkable moiety for interstrand crosslinking with a hybridized nucleic acid, and (2) an attachment moiety for attachment to a biological sample or a matrix. As illustrated in FIG. 1C, an example immobilization oligonucleotide can comprise a psoralen crosslinkable moiety and a C6 methacrylate. However, multiple different iterations of the immobilization oligonucleotide with different crosslinkable and attachment moieties disclosed herein could be used.

In an example, the hybridization region of the immobilization oligonucleotide is an oligodT sequence. The immobilization oligonucleotide can thus hybridize to a sequence in the polyA tail of a plurality of mRNA target nucleic acids. After fixation and permeabilization of the biological sample, the immobilization oligonucleotide is added to the tissue, allowed to hybridize and tile across the poly-A tails of the mRNA, washed, and exposed to UV light to crosslink the probe to the mRNA via the psoralen crosslinkable moiety.

Next, acrylamide monomers of a matrix-forming material are added to the biological sample, allowed to polymerize throughout the tissue and covalently incorporate the methacrylate moieties on the immobilization oligonucleotide into the hydrogel matrix. The mRNA is now covalently attached at several points along the poly-A tail into the matrix. Clearing with protease K and SDS removes the tissue components with the exception of very large (e.g., nucleus) structures, but the spatiality of the mRNA is intact.

A plurality of circular or circularizable primary probes or probe sets comprising target recognition sequences complementary to different target sequences in a plurality of different mRNAs are contacted with the sample and allowed to hybridize to their respective target sequence. Circularizable probes such as padlock probes can be circularized by ligation using the target sequences as templates, and rolling circle amplification can be performed to generate RCPs comprising barcode sequences corresponding to the target nucleic acids, which can then be detected at spatially localized positions in the sample or matrix.

Optionally, the RCP can also be attached to the matrix. For example, the matrix can be a multifunctional matrix (e.g., a multifunctional hydrogel), wherein the matrix comprises acrylamide monomers for attachment to the immobilization oligonucleotide, and a second anchoring moiety for attachment to the RCP (in one example, with methylsulfone linkers spiked into the matrix). In some embodiments, the matrix comprises acrylamide and methylsulfone. The ligated circularized probes cab be annealed to a thiolated primer and connected into the matrix via the methylsulfone linkers. Additionally, the rolling circle amplification can be performed with normal dNTPs with a bit of alpha-thiol nucleotides spiked in. These thiol nucleotides can be used by Phi29 as a substrate and further connected into the matrix mesh through the orthogonal methylsulfone moieties

In some experiments, the biological sample is contacted with an intermediate probe that hybridizes to the RCA product. The intermediate probe further comprises one or more binding regions for fluorescently-labeled probes. Once a signal associated with the RCA product is detected in one probe hybridization cycle, the intermediate probe and/or fluorescently-labeled probes can be dehybridized from the RCA product (e.g., by washing). The target nucleic acid remains immobilized biological sample via the interstrand crosslinking with the immobilization oligonucleotide and the attachment of the immobilization oligonucleotide to the biological sample or matrix. Multiple probe hybridization and dehybridization cycles can be performed to allow for decoding of the barcode sequence in the RCA product.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
(a) contacting a biological sample with an immobilization oligonucleotide functionalized with a crosslinkable moiety and an attachment moiety,
wherein the immobilization oligonucleotide comprises a hybridization region that hybridizes to a target nucleic acid in the biological sample; and
(b) crosslinking the crosslinkable moiety of the immobilization oligonucleotide to the hybridized target nucleic acid and attaching the attachment moiety to the biological sample or a matrix embedding the biological sample,
thereby immobilizing the target nucleic acid in the biological sample or the matrix.

2. The method of claim 1, wherein the method comprises (c) hybridizing a primary probe or probe set to a target sequence in the target nucleic acid.

3. The method of claim 2, wherein the method comprises (d) detecting the primary probe or probe set or a product of the primary probe or probe set associated with the target nucleic acid.

4. The method of any of claims 1-3, wherein the hybridization region comprises a sequence of at least 5, 10, 15, or 20 thymines.

5. The method of any of claims 1-4, wherein the hybridization region is an oligo deoxythymidine (oligo dT) sequence.

6. The method of any of claims 1-5, wherein the target nucleic acid is RNA, optionally wherein the target nucleic acid is mRNA.

7. The method of claim 6, wherein the target nucleic acid is an mRNA comprising a polyA tail, and the immobilization oligonucleotide hybridizes to the target nucleic acid at the polyA tail.

8. The method of claim 7, wherein the method comprises hybridizing multiple copies of the immobilization oligonucleotide to the polyA tail.

9. The method of any of claims 1-8, wherein the target nucleic acid is an RNA fragment.

10. The method of claim 9, wherein the RNA fragment does not comprise a polyA tail.

11. The method of any of claims 1-3 and 6-10, wherein the hybridization region is a random sequence and/or comprises universal bases.

12. The method of claim 11, wherein the hybridization region is a sequence of universal bases.

13. The method of any of claims 1-12, wherein the method comprises hybridizing multiple immobilization oligonucleotides to the target nucleic acid.

14. The method of any of claims 1-13, wherein the immobilization oligonucleotide is not capable of being extended by a polymerase.

15. The method of claim 14, wherein the immobilization oligonucleotide comprises a 3′ dideoxynucleotide.

16. The method of any of claims 1-15, wherein the crosslinkable moiety is a modified nucleoside in the immobilization oligonucleotide or is connected to a nucleotide residue in the hybridization region of the immobilization oligonucleotide.

17. The method of any of claims 1-16, wherein crosslinking occurs between the hybridization region of the immobilization oligonucleotide and the hybridized target nucleic acid.

18. The method of any of claims 1-17, wherein the crosslinkable moiety is configured to crosslink to a nucleobase of the hybridized target nucleic acid.

19. The method of any of claims 1-18, wherein the method comprises irradiating the biological sample or the matrix to photo-activate the crosslinkable moiety.

20. The method of claim 18, wherein the biological sample or the matrix is irradiated using a 350-400 nm wavelength of light.

21. The method of any of claims 18-20, wherein the nucleobase is a thymine, uridine, or cytosine.

22. The method of any of claims 18-20, wherein the nucleobase is an adenine.

23. The method of any of claims 16-22, wherein the crosslinkable moiety is connected to the nucleotide residue via a linker.

24. The method of any of claims 1-23, wherein the crosslinkable moiety is a vinylcarbazone-based moiety.

25. The method of any of claims 1-24, wherein the crosslinkable moiety is a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D).

26. The method of any of claims 1-25, wherein the crosslinkable moiety is a 3-cyanovinylcarbazole phosphoramidite or a pyranocarbazole phosphoramidite.

27. The method of any of claims 1-24, wherein the crosslinkable moiety is a psoralen or a psoralen derivative, optionally wherein the psoralen is a C2 psoralen.

28. The method of any of claim 27, wherein the crosslinkable moiety is a psoralen C2 phosphoramidite.

29. The method of any of claims 1-24, wherein the crosslinkable moiety is a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite).

30. The method of any of claims 1-24, wherein the crosslinkable moiety is a 5′-Dimethoxytrityl-5-iodo-2′-deoxyUridine,3′-[(2-cyanoethyl)-(N,N-diisopropyl)]-phosphoramidite (5-I-dU-CE phosphoramidite).

31. The method of any of claims 1-30, wherein the immobilization oligonucleotide comprises two or more nucleotide residues functionalized with crosslinkable moieties in the hybridization region.

32. The method of any of claims 1-31, wherein the immobilization oligonucleotide comprises three, four, five, or more nucleotide residues functionalized with crosslinkable moieties in the hybridization region.

33. The method of any of claims 1-32, wherein the hybridization region comprises one or more universal bases.

34. The method of any of claims 16-33, wherein the nucleotide residue comprising or connected to the crosslinkable moiety comprises a universal base.

35. The method of any of claims 31-34, wherein the nucleotide residues connected to the crosslinkable moieties comprise universal bases.

36. The method of any of claims 33-35, wherein the one or more universal bases comprise a pseudouridine and/or an inosine.

37. The method of claim 36, wherein the universal base is pseudouridine.

38. The method of any of claims 1-37, wherein the attachment moiety is an amine, a thiol, an azide, an alkyne, a nitrone, an alkene, a tetrazine, a tetrazole, an acrydite or other click reactive group.

39. The method of any of claims 1-38, wherein the attachment moiety is attached to an anchoring moiety in the biological sample or the matrix, wherein the attachment moiety and the anchoring moiety are a ligand-ligand binding pair, or functional moieties that can react with each other.

40. The method of any of claims 1-39, wherein the attachment moiety is an acrydite moiety, optionally wherein the acrydite is a C6 methacrylate.

41. The method of any of claims 1-40, wherein the attachment moiety is a methacrylate C6 phosphoramidite.

42. The method of any of claims 2-41, wherein the primary probe or probe set is a circular probe or a circularizable probe or probe set.

43. The method of claim 42, wherein the method comprises circularizing the circularizable probe or probe set to generate a circularized probe.

44. The method of claim 43, wherein the method comprises performing rolling circle amplification of the circular or circularized probe to generate a rolling circle amplification product (RCP).

45. The method of claim 44, wherein the rolling circle amplification is performed using a primer comprising a functional moiety for attachment to the biological sample or the matrix.

46. The method of claim 45, wherein the functional moiety of the primer is orthogonal to the attachment moiety of the immobilization oligonucleotide.

47. The method of any of claims 44-46, wherein the method comprises contacting the biological sample or the matrix with a nucleotide mixture comprising one or more modified crosslinkable nucleotides for incorporation into the RCP.

48. The method of any of claims 45-47, wherein the method comprises crosslinking the functional moiety of the primer and/or the one or more modified crosslinkable nucleotide residues in the RCP to the biological sample or the matrix.

49. The method of any of claims 44-48, wherein the detecting in (d) comprises detecting the RCP.

50. The method of claim 49, wherein detecting the RCP comprises binding an intermediate probe directly or indirectly to the RCP, binding a detectably labeled probe directly or indirectly to a detection region of the intermediate probe, and detecting a signal associated with the detectably labeled probe.

51. The method of claim 50, wherein the method comprises performing one or more wash steps to remove unbound and/or nonspecifically bound intermediate probe molecules from the primary probes or the products of the primary probes.

52. The method of any of claims 1-51, wherein the detecting in (d) comprises:
detecting signals associated with detectably labeled probes that are hybridized to barcode regions or complements thereof in the primary probe or probe set or a product thereof; and/or
detecting signals associated with detectably labeled probes that are hybridized to intermediate probes which are in turn hybridized to the barcode regions or complements thereof.

53. The method of claim 52, wherein the detectably labeled probes are fluorescently labeled.

54. The method of any of claims 1-43, wherein the detecting in (d) comprises binding an intermediate probe directly or indirectly to the primary probe or probe set, binding a detectably labeled probe directly or indirectly to a detection region of the intermediate probe, and detecting a signal associated with the detectably labeled probe.

55. The method of any of claims 1-43, wherein detecting the primary probe or probe set comprises amplifying a signal associated with the primary probe or probe set, wherein amplifying the signal comprises RCA of a probe that directly or indirectly binds to the primary probe or probe set and/or the amplification product thereof; hybridization chain reaction (HCR) directly or indirectly on the primary probe or probe set and/or the amplification product thereof; linear oligonucleotide hybridization chain reaction (LO-HCR) directly or indirectly on the primary probe or probe set and/or the amplification product thereof; primer exchange reaction (PER) directly or indirectly on the primary probe or probe set and/or the amplification product thereof; assembly of branched structures directly or indirectly on the primary probe or probe set and/or the amplification product thereof; hybridization of a plurality of detectable probes directly or indirectly on the primary probe or probe set and/or the amplification product thereof, or any combination thereof.

56. The method of any one of claims 1-55, wherein the method comprises contacting the sample with a matrix-forming material and using the matrix-forming material to form the matrix.

57. The method of any one of claims 39-56, wherein the matrix is functionalized with the anchoring moiety to bind covalently or non-covalently to the attachment moiety.

58. The method of any of claims 39-57, wherein the anchoring moiety is a reactive group selected from the group consisting of acrydite, NHS ester, azide, maleimide, amine, and carboxyl groups.

59. The method of any of claims 1-58, wherein the attachment moiety is a first attachment moiety, and the immobilization oligonucleotide comprises a second attachment moiety and the method comprises attaching the second attachment moiety to the biological sample or a matrix embedding the biological sample, optionally wherein the second attachment moiety is different from the first attachment moiety.

60. The method of claim 59, wherein the second attachment moiety is an amine, a thiol, an azide, an alkyne, a nitrone, an alkene, a tetrazine, a tetrazole, an acrydite or other click reactive group.

61. The method of claim 59, wherein the second attachment moiety is a photo-crosslinkable nucleotide.

62. The method of claim 61, wherein the second attachment moiety is 5-bromo deoxyuridine (BrdU).

63. The method of claim 59 or 60, wherein the second attachment moiety is attached to a second anchoring moiety in the biological sample or a matrix embedding the biological sample, wherein the second attachment moiety and the second anchoring moiety are a ligand-ligand binding pair, or functional moieties that can react with each other.

64. The method of claim 63, wherein the first attachment moiety and the second attachment moiety are attached to the same matrix embedding the biological sample using orthogonal reaction chemistries.

65. The method of claim 63, wherein the first attachment moiety is attached to a first matrix embedding the biological sample and the second attachment moiety is attached to a second matrix embedding the biological sample, optionally wherein the first and second matrix are intertwined.

66. A method of analyzing a biological sample, comprising:
(a) performing an extension reaction of a primary probe hybridized to a target nucleic acid in the biological sample to incorporate one or more nucleotides functionalized with an attachment moiety using the target nucleic acid as a template, thereby forming a primary immobilizable probe;
(b) attaching the attachment moiety to the biological sample or a matrix embedding the biological sample; and
(c) detecting the attached primary immobilizable probe at a position in the biological sample or the matrix.

67. The method of claim 66, wherein the primary probe comprises an overhang region at its 5′ end.

68. The method of claim 66 or 67, wherein the method comprises performing extension reactions of a plurality of primary probes hybridized to the target nucleic acid.

69. The method of claim 68, wherein the extension reactions are to incorporate one or more nucleotides functionalized with an attachment moiety using the target nucleic acid as a template into the plurality of primary probes, optionally wherein the attachment moiety is a crosslinkable moiety.

70. The method of claim 68 or 69, wherein each primary probe of the plurality of primary probes comprises an overhang region at its 5′ end.

71. The method of any of claims 67-70, wherein the method comprises hybridizing a detection probe to the overhang region and detecting the detection probe or a product thereof.

72. The method of claim 71, wherein the detection probe comprises (i) a recognition sequence that hybridizes to a sequence of the overhang region and (ii) a reporter sequence for binding directly or indirectly to a detectably labeled probe.

73. The method of any of claims 71-72, wherein the detection probe is a first detection probe, and the method comprises removing the first detection probe after detecting the first detection probe, hybridizing a second detection probe to the overhang region, and detecting the second detection probe.

74. The method of claim 72, wherein the detection probe is a circular or circularizable probe, and the method comprises performing rolling circle amplification of the detection probe and detecting a product thereof.

75. The method of claim 71 or claim 73, wherein the detection probe is a detectably labeled probe.

76. The method of any of claims 68-75, wherein the extension reactions are performed simultaneously.

77. The method of any of claims 66-76, wherein the extension reaction or extension reactions are performed using a polymerase lacking strand displacing activity.

78. The method of any of claims 68-77, wherein the extension reaction of a primary probe of the plurality of primary probes does not displace other primary probes of the plurality of primary probes from the target nucleic acid.

79. The method of any of claims 66-78, wherein the extension reaction or extension reactions are performed for less than 30 minutes, less than 10 minutes, or less than 5 minutes.

80. A method of analyzing a biological sample, comprising:
(a) contacting the biological sample with a primary probe;
(b) hybridizing a secondary immobilizable probe to the primary probe to form an immobilizable probe complex, wherein the secondary immobilizable probe is functionalized with a crosslinkable moiety and an attachment moiety,
(c) crosslinking the crosslinkable moiety of the immobilization oligonucleotide to the hybridized primary probe and attaching the attachment moiety to the biological sample or a matrix embedding the biological sample, thereby forming a crosslinked probe complex, and
(d) hybridizing a detection probe to the primary probe and detecting the detection probe or a product thereof, thereby detecting the crosslinked probe complex at a position in the biological sample or the matrix.

81. The method of claim 80, wherein (b) comprises hybridizing a plurality of secondary immobilizable probes to the primary probe to form the immobilizable probe complex, wherein each secondary immobilizable probe comprises an attachment moiety, and wherein the method comprises, using the attachment moiety, attaching each secondary immobilizable probe to the biological sample or the matrix to form the crosslinked probe complex.

82. The method of claim 80 or 81, wherein the secondary immobilizable probe or plurality thereof hybridizes to an overhang region of the primary probe.

83. The method of any of claims 80-82, wherein the secondary immobilizable probe or plurality thereof does not comprise a detectable label.

84. The method of any of claims 80-83, wherein the detection probe comprises (i) a recognition sequence that hybridizes to a sequence of the overhang region of the primary probe, and (ii) a reporter sequence for binding directly or indirectly to a detectably labeled probe.

85. The method of any of claims 80-84, wherein the detection probe is a first detection probe, and the method comprises removing the first detection probe after detecting the first detection probe, hybridizing a second detection probe to the overhang region, and detecting the second detection probe.

86. The method of any of claims 80-85, wherein the detection probe is a circular or circularizable probe, and the method comprises performing rolling circle amplification of the detection probe and detecting a product thereof.

87. The method of any of claims 80-86, wherein the detection probe is a detectably labeled probe.

88. The method of any of claims 1-87, wherein the method further comprises hybridizing a tertiary immobilizable probe comprising an attachment moiety to the secondary probe or plurality thereof.

89. The method of claim 88, wherein the method further comprises crosslinking the tertiary immobilizable probe to the biological sample or the matrix to form the crosslinked probe complex.

90. The method of any of claims 80-89, wherein the crosslinkable moiety is a modified nucleoside in the immobilization oligonucleotide or is connected to a nucleotide residue in the hybridization region of the immobilization oligonucleotide.

91. The method of any of claims 80-90, wherein crosslinking occurs between the hybridization region of the immobilization oligonucleotide and the hybridized target nucleic acid.

92. The method of any of claims 80-91, wherein the crosslinkable moiety is configured to crosslink to a nucleobase of the hybridized target nucleic acid.

93. The method of any of claims 80-92, wherein the method comprises irradiating the biological sample or the matrix to photo-activate the crosslinkable moiety.

94. The method of claim 93, wherein the biological sample or the matrix is irradiated using a 350-400 nm wavelength of light.

95. The method of any of claims 92-94, wherein the nucleobase is a thymine, uridine, or cytosine.

96. The method of any of claims 92-94, wherein the nucleobase is an adenine.

97. The method of any of claims 90-96, wherein the crosslinkable moiety is connected to the nucleotide residue via a linker.

98. The method of any of claims 80-97, wherein the crosslinkable moiety is a vinylcarbazone-based moiety.

99. The method of any of claims 80-98, wherein the crosslinkable moiety is a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D).

100. The method of any of claims 80-99, wherein the crosslinkable moiety is a 3-cyanovinylcarbazole phosphoramidite or a pyranocarbazole phosphoramidite.

101. The method of any of claims 80-98, wherein the crosslinkable moiety is a psoralen or a psoralen derivative, optionally wherein the psoralen is a C2 psoralen.

102. The method of any of claim 101, wherein the crosslinkable moiety is a psoralen C2 phosphoramidite.

103. The method of any of claims 80-98, wherein the crosslinkable moiety is a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite).

104. The method of any of claims 80-98, wherein the crosslinkable moiety is a 5′-Dimethoxytrityl-5-iodo-2′-deoxyUridine,3′-[(2-cyanoethyl)-(N,N-diisopropyl)]-phosphoramidite (5-I-dU-CE phosphoramidite).

105. The method of any of claims 80-104, wherein the immobilization oligonucleotide comprises two or more nucleotide residues functionalized with crosslinkable moieties in the hybridization region.

106. The method of any of claims 80-105, wherein the immobilization oligonucleotide comprises three, four, five, or more nucleotide residues functionalized with crosslinkable moieties in the hybridization region.

107. The method of any of claims 80-106, wherein the hybridization region comprises one or more universal bases.

108. The method of any of claims 90-107, wherein the nucleotide residue comprising or connected to the crosslinkable moiety comprises a universal base.

109. The method of any of claims 105-108, wherein the nucleotide residues connected to the crosslinkable moieties comprise universal bases.

110. The method of any of claims 80-109, wherein the secondary immobilizable probe or plurality thereof comprises a universal hybridization region comprising (i) one or more universal or random bases and (ii) the crosslinkable moiety.

111. The method of claim 110, wherein the universal hybridization region of the secondary immobilizable probe or plurality thereof hybridizes non-specifically to the primary probe.

112. The method of any of claims 80-111, wherein the primary probe is hybridized to a target nucleic acid in the sample.

113. The method of claim 112, wherein the primary probe is crosslinked to the target nucleic acid, biological sample, and/or the matrix.

114. A method of analyzing a tissue sample, comprising:
(a) contacting the tissue sample with a primary immobilizable probe, wherein the primary immobilizable probe comprises a hybridization region capable of hybridizing to a region of interest in a target nucleic acid and an attachment moiety;
(b) using the attachment moiety, crosslinking the primary immobilizable probe a matrix embedding the biological sample;
(c) clearing the tissue sample;
(d) hybridizing a detection probe to a barcode sequence in the primary immobilizable probe; and
(e) detecting the detection probe or a product thereof at a position in the biological sample or the matrix.

115. The method of claim 114, wherein the detection probe comprises (i) a recognition sequence that hybridizes to a sequence of the overhang region of the primary probe, and (ii) a reporter sequence for binding directly or indirectly to a detectably labeled probe.

116. The method of claim 114 or 115, wherein the detection probe is a first detection probe, and the method comprises removing the first detection probe after detecting the first detection probe, hybridizing a second detection probe to the overhang region, and detecting the second detection probe.

117. The method of any of claims 114-115, wherein the detection probe is a circular or circularizable probe, and the method comprises performing rolling circle amplification of the detection probe and detecting a product thereof.

118. The method of any of claims 114-117, wherein the detection probe is a detectably labeled probe.

119. A method of analyzing a biological sample, comprising:
(a) contacting the biological sample with an immobilization oligonucleotide and a primary probe, wherein the primary probe hybridizes to a target nucleic acid in the biological sample, and wherein the immobilization oligonucleotide comprises an attachment moiety;
(b) ligating the primary probe to the immobilization oligonucleotide to form a ligated immobilizable probe comprising the primary probe and the immobilization oligonucleotide;
(c) crosslinking the attachment moiety of the ligated immobilizable probe to a matrix embedding the biological sample, thereby crosslinking the immobilizable probe to the matrix;
(d) contacting the biological sample with a detection probe that hybridizes to the ligated immobilizable probe or a product thereof; and
(e) detecting the detection probe or a product of the detection probe at a position in the biological sample.

120. The method of claim 119, wherein the detection probe hybridizes to a detection probe hybridization sequence in a first overhang region of the primary probe in the immobilizable probe.

121. The method of claim 119 or 120, wherein the method comprises contacting the immobilization oligonucleotide with a splint that hybridizes to at least a portion of the primary probe and at least a portion of the immobilization oligonucleotide.

122. The method of claim 121, wherein the splint serves as a template for ligating the primary probe to the immobilization oligonucleotide.

123. The method of claim 121 or 122, wherein the splint hybridizes to a splint hybridization sequence in a second overhang region of the primary probe.

124. The method of any of claims 119-123, wherein the immobilization oligonucleotide and the primary probe hybridize to adjacent sequences of the target nucleic acid.

125. The method of any of claims 119-124, wherein the target nucleic acid serves as a template for ligating the primary probe to the immobilization oligonucleotide.

126. A method of analyzing a biological sample, comprising:
(a) contacting the biological sample with a probe or probe set comprising a first hybridization region and a second hybridization region, wherein the first hybridization region and the second hybridization region hybridize to a first and second target sequence, respectively, in a target nucleic acid, wherein the first and second target sequences are 3′ and 5′, respectively, to a first sequence of a region of interest in the target nucleic acid, wherein the region of interest comprises a first nucleobase;
(b) contacting the biological sample with a crosslinkable nucleotide complementary to the first nucleobase;
(c) extending the 3′ end of the first hybridization region with a polymerase using the first sequence of the region of interest as a template, thereby incorporating the crosslinkable nucleotide into the first hybridization region;
(d) ligating the extended 3′ end of the first hybridization region and the 5′ end of the second hybridization region to form a ligated probe;
(e) crosslinking the incorporated crosslinkable nucleotide to the biological sample or a matrix embedding the biological sample; and
(f) detecting the crosslinked ligated probe or a product thereof at a location in the biological sample or the matrix.

127. The method of claim 126, wherein the ligatable probe or probe set is a ligatable probe set comprising a first part and a second part, wherein the first part comprises the first hybridization region and the second part comprises the second hybridization region.

128. The method of claim 127, wherein detecting the crosslinked ligated probe comprises detecting a sequence in an overhang region of the first part and/or second part.

129. The method of any of claims 126-128, wherein the ligatable probe or probe set is a circularizable probe or probe set.

130. A method of analyzing a biological sample, comprising:
(a) contacting the biological sample with a circularizable probe comprising (i) a 3′ arm that hybridizes to a first target sequence in a target nucleic acid in the biological sample, and (ii) a 5′ arm that hybridizes to a second target sequence in the target nucleic acid, wherein the first and second target sequence are 3′ and 5′, respectively, to a first sequence of a region of interest comprising a first nucleobase;
(b) contacting the biological sample with a crosslinkable nucleotide complementary to the first nucleobase;
(c) extending the 3′ arm of the circularizable probe with a polymerase using the first sequence of the region of interest as a template, thereby incorporating the crosslinkable nucleotide into the circularizable probe;
(d) ligating the extended 3′ arm and the 5′ arm of the circularizable probe to form a circularized probe;
(e) crosslinking the incorporated crosslinkable nucleotide to the biological sample or to a matrix embedding the biological sample; and
(f) detecting the crosslinked circularized probe or a product thereof at a location in the biological sample or the matrix.

131. The method of any of claims 126-130, wherein an alternative sequence of the region of interest does not comprise the first nucleobase, such that the crosslinkable nucleotide is not incorporated into the circularizable probe when using the alternative sequence of the region of interest as a template.

132. The method of claim 130 or 131, wherein detecting the crosslinked circularized probe or a product thereof comprises performing rolling circle amplification (RCA) using the circularized probe as a template to form a rolling circle amplification product (RCP) and detecting the RCP in the sample.

133. The method of claim 132, wherein the method comprises decrosslinking the circularized probe prior to performing RCA.

134. The method of any of claims 132-133, wherein the method comprises
(a) hybridizing a secondary circular probe to the crosslinked circularized probe, or hybridizing a secondary circularizable probe to the crosslinked circularized probe and circularizing the hybridized secondary circularizable probe to generate a secondary circularized probe, and
(b) performing RCA using the secondary circular probe or secondary circularized probe as a template to form a rolling circle amplification product (RCP) and detecting the RCP in the sample.

135. The method of any of claims 126-134, wherein the target nucleic acid is a cDNA.

136. The method of any of claims 56-135, wherein the matrix-forming material is a first species of matrix-forming material, wherein the method comprises polymerizing the first species of matrix-forming material to form a first matrix, contacting the first matrix with a second species of matrix-forming material, and polymerizing the second species of matrix-forming material to form a second matrix.

137. The method of claim 136, wherein the method comprises polymerizing the second species of matrix-forming material to form the second matrix after hybridizing a primary probe or probe set to a target sequence in the target nucleic acid.

138. The method of claim 136 or 137, wherein the method comprises contacting the first matrix with the second species of matrix-forming material after hybridizing a primary probe or probe set to a target sequence in the target nucleic acid.

139. The method of any of claims 1-135, wherein the matrix is a hydrogel matrix.

140. The method of any of claims 136-138, wherein the first matrix is a hydrogel matrix and/or the second matrix is a hydrogel matrix.

141. The method of any of claims 1-140, wherein the biological sample is non-homogenized and optionally selected from the group consisting of a formalin-fixed, paraffin-embedded (FFPE) sample, a frozen tissue sample, and a fresh tissue sample.

142. The method of any of claims 1-141, wherein the biological sample is permeabilized.

143. The method of any of claims 1-142, wherein the biological sample is embedded in a matrix, optionally wherein the matrix comprises a hydrogel.

144. The method of any of claims 1-143, wherein the biological sample is cleared.

145. The method of any of claims 1-144, wherein the method comprises clearing the biological sample after crosslinking the crosslinkable moiety to the target nucleic acid and attaching the attachment moiety to the matrix, optionally wherein the clearing comprises contacting the biological sample with a proteinase.

146. The method of any of claims 1-145, wherein the biological sample is a tissue slice between about 1 μm and about 50 μm in thickness, optionally wherein the tissue slice is between about 5 μm and about 35 μm in thickness.
